SEQUENCE LISTING (3 sequences)

SEQ ID NO: 1 - DNA, 33 bp, Artificial Sequence
  Synthetic Construct
tcagcctgttaacctgactgttcgatatattca

SEQ ID NO: 2 - DNA, 33 bp, Artificial Sequence; identical to SEQ ID NO: 1
  Synthetic Construct

SEQ ID NO: 3 - DNA, 35 bp, Artificial Sequence
  Synthetic Construct
tttgatgatatcccgtttcaggaaatcaacatgtc

(19) United States
(12) Patent Application Publication
(10) Pub. No.: US 2013/0196341 A1
(43) Pub. Date: Aug. 1, 2013

(54) METHODS AND COMPOSITIONS FOR DETECTION OF ANALYTES

(71) Applicants: Lori Anne Neely, Reading, MA (US); Brian M. Mozeleski, Knox, ME (US); Mark John Audeh, Brighton, MA (US); Jordan R. Raphel, Dedham, MA (US); Thomas Jay Lowery, JR., Belmont, MA (US)

(72) Inventors: Lori Anne Neely, Reading, MA (US); Brian M. Mozeleski, Knox, ME (US); Mark John Audeh, Brighton, MA (US); Jordan R. Raphel, Dedham, MA (US); Thomas Jay Lowery, JR., Belmont, MA (US)

(73) Assignee: T2 Biosystems ,Inc., Lexington, MA (US)

(21) Appl. No.: 13/808,347

(22) PCT Filed: Jul. 6, 2011
(86) PCT No.: PCT/US2011/043037

§ 371 Date: Apr. 8, 2013

Related U.S. Application Data
(60) Provisional application No. 61/361,766, filed on Jul. 6, 2010.

(51) Int. Cl. G01N 33/53

(52) U.S. Cl. CPC G01N 33/5308; G01N 33/53; G01N 33/5304
USPC 435/7.1; 436/501; 435/288.7; 422/554

(57) ABSTRACT

Disclosed are methods and compositions for detecting analytes, including proteins, polysaccharides, viruses, nucleic acids and cells. The methods and compositions utilize a reporter probe, suitably a multivalent reporter probe, to detect the presence of the analytes. The methods and compositions can be used for non-enzymatic detection of nucleic acids.

### CROSS REFERENCE TO RELATED APPLICATIONS

This application claims benefit of U.S. Provisional Patent Application No. 61/361,766, filed Jul. 6, 2010, which is hereby incorporated by reference in its entirety.

### GRANT SUPPORT

This invention was made with Government support under Contract No. 2004*H838109*000 awarded by the Central Intelligence Agency. The Government has certain rights in this invention.

### BACKGROUND OF THE INVENTION

1. Field of the Invention

The present invention relates to methods and compositions for detecting analytes, including proteins, nucleic acids, saccharides, lipids, small molecules, ions, gases, infectious agents, and cells, in a sample. The present invention enables the detection of analytes without the need for enzymatic or cell-based amplification methods, such as are currently used for the detection of nucleic acids.

2. Background

Particle-based methods for sensitive and selective detection of oligonucleotides have been described and demonstrated by others to identify target sequence presence, and/or to select between target sequences that differ by a single-base substitution, insertion, or deletion. See, e.g., Elghanian, R., et al., “Selective Colorimetric Detection of Polynucleotides Based on the Distance-Dependent Optical Properties of Gold Nanoparticles,” Science 277:1078-1081 (1997); Nam, J.-M., et al., “Bio-Bar-Code-Based DNA Detection with PCR-like Sensitivity,” J. Am. Chem. Soc. 126:5932-5933 (2004); Storhoff, J. J., et al., “One-Pot Colorimetric Differentiation of Polynucleotides with Single Base Imperfections Using Gold Nanoparticle Probes,” J. Am. Chem. Soc. 120:1959-1964 (1998); Hill, H. D., et al., “Nonenzymatic detection of bacterial genomic DNA using the bio bar code assay,” Anal. Chem. 79:9218-23 (2007); and Rosi, N. L., et al., “Nanostructures in Biodiagnostics,” Chem. Rev. 105:1547-1562 (2005). However, detection of bacterial genomic DNA using such methods has a lower detection limit almost four orders of magnitude greater than the detection of oligonucleotides (i.e., about 2.5 fM). Thus, achieving attomolar (“aM”) or femtomolar (“fM”) sensitivity levels in clinical practice is unlikely using these methods.

Polymerase chain reaction (PCR) based approaches have the highest sensitivity of all current methods for detecting nucleic acids. See Anal. Chem. 79:9218-9223 (2007). Assays that detect genomic DNA at aM concentrations typically amplify a target by PCR. See Jochen, W., et al., “Real-Time Polymerase Chain Reaction,” ChemBioChem 4:1120-1128 (2003), and Valasek, M. A., et al., “The power of real-time PCR,” Advan. Physiol. Edu. 29:151-159 (2005). In theory, PCR methods can amplify and detect the presence of a single copy of a nucleic acid analyte. Detection of five or fewer copies of a DNA sequence in a sample has been demonstrated. Id. The power of PCR-based techniques lies in signal amplification afforded by the polymerase chain reaction, which roughly doubles the amount of target molecule with each cycle. Over many cycles, increased concentration of target molecules becomes sufficient for even low-sensitivity secondary assay detection techniques (e.g., ethidium bromide gel electrophoresis). However, use of enzyme based amplification strategies imposes constraints (e.g., temperature, pressure, humidity, costs, reagent stability, sample preparation before amplification, contamination etc.) on conditions for carrying out detection methods and assays. Further, samples containing nucleic acids may have substances present that may inhibit amplification leading to sample-to-sample variability. Thus, non-enzymatic amplification and detection methods which are not restrained by such circumstances would be beneficial to allow for universal applications. In addition, methods for determining the presence and concentration of analytes that allow for detection without requiring sample processing and clean-up are needed. The present invention provides these needs.

### BRIEF SUMMARY OF THE INVENTION

The present invention provides methods of detecting one or more analytes in a sample. In some embodiments, the methods comprise contacting a sample with a reporter particle capable of binding to the analyte, wherein in the presence of the analyte the reporter particle binds to the analyte. Unbound reporter particles are removed from the sample, and the sample is contacted with a detector moiety, wherein in the presence of the remaining reporter particle, the detector moiety forms an agglomerate. Therefore, one or more analytes are detected by measuring the value of a property of the agglomerate. Furthermore, the value of a sample comprising one or more analytes differs from the value of a reference sample lacking the one or more analytes. Thus, comparing a property of a sample with a reference that lacks the analyte can provide a quantitative measurement of analyte concentration in a sample.

Analytes suitable for detection by the methods of the present invention include, but are not limited to, proteins, nucleic acids, saccharides, lipids, small molecules, ions, gases, infectious agents, cells, and combinations thereof.

In some embodiments, the presence of one or more analytes in a sample is detected by measuring a property of a sample selected from: a nuclear magnetic resonance property, a relaxation time, an ultraviolet absorption, a visible absorption, a fluorescence intensity, a fluorescence decay time, a circular dichroism, a radioactive half-life, a radioactive emission signal, a turbidity, a density, and combinations thereof.

In some embodiments, detecting comprises determining a relaxation time of the sample by magnetic resonance spectroscopy. In some embodiments, detecting comprises determining a T2 relaxation time of a sample.

In some embodiments, a detector moiety is magnetic, light-absorptive, fluorescent, chiral, radioactive, or a combination thereof. In some embodiments, in the presence of an analyte, the detector moiety binds to the analyte. In some embodiments, a detector moiety comprises a binding group capable of binding to a reporter particle, wherein in the presence of remaining reporter particle the detector moiety binds to a remaining reporter particle. In some embodiments, a detector moiety comprises a magnetic particle, wherein in the presence of the reporter particle an agglomerate of the magnetic particles is formed.

In some embodiments, a reporter particle comprises a non-magnetic reporter particle that includes a plurality of binding groups. In some embodiments, reporter particles that are not bound to an analyte are removed by washing a sample.

In some embodiments, at least a detector moiety, a reporter particle, a capture particle, or a combination thereof is magnetic (e.g., paramagnetic or superparamagnetic). In some embodiments, a sample comprising a paramagnetic or superparamagnetic species is subjected to magnetic assisted agglomeration prior to the detecting.

In some embodiments, the methods comprise contacting a sample with a non-magnetic reporter particle comprising a plurality of binding groups capable of binding to a first target site on one or more analytes, wherein in the presence of an analyte the non-magnetic reporter particle binds to a first target site on an analyte. Non-magnetic reporter particles that are not bound to an analyte are removed. The methods comprise contacting the sample (comprising [analyte]-[non-magnetic reporter particle] conjugates) with a plurality of detector moieties comprising magnetic particles, wherein in the presence of the reporter particle, the non-magnetic reporter particles form an agglomerate with the magnetic detector particles. The analyte is quantitatively detected by a change in a signal corresponding to a relaxation time of the sample when the analyte is present compared to a relaxation time of a reference lacking the analyte.

In some embodiments, after contacting a sample with a non-magnetic reporter particle comprising a plurality of binding groups capable of binding to a first target site on one or more analyte/s, the sample is contacted with a plurality of magnetic capture particles capable of binding to a second target site on the analyte, wherein in the presence of the analyte the magnetic capture particles bind to the second target site on the analyte, and wherein in the presence of the reporter particle, the non-magnetic reporter particle form an agglomerate with the magnetic capture particles. Magnetic reporter particles that are not bound to an analyte are removed from the sample to provide a complex comprising analytes bound to both magnetic capture particles and non-magnetic reporter particles. The analyte is detected in the sample by measuring a property such as a relaxation time and comparing the property with that of a reference sample lacking the analyte (e.g., a change in relaxation time for a sample containing an analyte compared to the relaxation time of a reference sample lacking an analyte).

The present invention is also directed to a method of detecting one or more analytes in a sample, the method comprising contacting the sample with a capture particle comprising a first binding group capable of specifically binding to a first binding site on the one or more analytes, wherein in the presence of an analyte, the capture particle binds to the first binding site; contacting the sample with a reporter particle comprising a plurality of binding groups capable of binding to the analyte-capture particle complex, wherein in the presence of the analyte, the reporter particle binds to the analyte-capture particle complex; removing unbound reporter particle from the sample; and detecting the presence of the reporter particle.

In some embodiments, a capture particle is magnetic. In some embodiments, an analyte bound to a magnetic capture particle is separated from the sample using a magnetic field.

In some embodiments, a method comprises disassociating a bound reporter particle from an analyte prior to the detecting. In some embodiments, a method comprises disassociating a bound reporter particle from an analyte after the removing and prior to the detecting.

Disassociating can include a process selected from: temperature denaturing, generating a pH gradient, reducing disulfide bonds, oxidizing disulfide bonds, mechanically disrupting, and combinations thereof. In some embodiments, a method comprises disassociating a target probe from an analyte by disrupting a specific binding interaction between a first binding group of a target probe and a first binding site on an analyte. The method can further include the step of, prior to the detecting, contacting the disassociated reporter particle with a detector moiety to form an aggregate of the reporter particle and the detector moiety, wherein the detecting includes measuring a value of a property of the aggregate, wherein the value of a sample including the one or more analytes differs from the value of a reference sample lacking the one or more analytes.

In one embodiment of any of the above methods, the sample is contacted with a target probe comprising a first binding group capable of specifically binding to one or more species in the sample. In some embodiments, a target probe comprises two or more binding groups that differ, and the target probe can specifically bind to two or more species in a sample. Target probes suitable for use with the present invention can include a binding group capable of specifically binding to an analyte, a reporter particle, a detector moiety, and/or a capture particle by specific binding interactions. For example, a method of the present invention can include contacting a sample with a target probe capable of specifically binding to one or more analytes and a reporter particle, separating unbound target probe from target probe bound to an analyte-capture particle complex, and dissociating the bound reporter particle from the analyte-capture particle complex prior to the step of detecting. In some embodiments, a target probe binds to a capture particle and a reporter particle via specific binding interactions with each of these species. In some embodiments, a target probe binds to a reporter particle and a detector moiety via specific binding interactions with each of these species.

The present invention also provides methods of detecting one or more analytes in a sample wherein the analytes comprise target nucleic acids. Thus, the present invention is directed to methods comprising contacting a sample with a magnetic capture particle comprising a first oligonucleotide complementary to a first nucleic acid sequence of a target nucleic acid, wherein in the presence of the target nucleic acid, the magnetic capture particle binds to the first nucleic acid sequence. The sample is contacted with a target probe comprising an oligonucleotide complementary to, and capable of binding with, a second nucleic acid sequence of the target nucleic acid, wherein the first and second nucleic acid sequences are different, and wherein in the presence of the target nucleic acid, the target probe binds to the second nucleic acid sequence. The sample is contacted with a reporter particle comprising a plurality of binding groups capable of binding to the target probe, wherein in the presence of the target nucleic acid, the reporter particle binds to the target probe. Reporter particles that are not bound to target nucleic acids are removed to provide a complex comprising the target nucleic acid bound to magnetic capture particles and reporter particles. The reporter particle is caused to disassociate from the target nucleic acid, and the presence of the reporter particle that was previously bound to the target nucleic acid is then detected.

The present invention is also directed to methods of detecting one or more target nucleic acids in a sample comprising target and non-target nucleic acids, the methods comprising contacting a sample with a magnetic capture particle comprising an oligonucleotide complementary to a first nucleic acid sequence of the target nucleic acid, wherein in the presence of the target nucleic acid, the magnetic capture particle binds to the first nucleic acid sequence via nucleotide base pairing. The sample is contacted with a target probe comprising an oligonucleotide complementary to a second nucleic acid sequence of the target nucleic acid, wherein in the presence of the target nucleic acid, the target probe binds to the second nucleic acid sequence via nucleotide base pairing, and a complex comprising the magnetic capture particle, the target nucleic acid and the target probe is formed, and wherein the first nucleic acid sequence and the second nucleic acid sequence are different. Non-target nucleic acids and unbound target probes are removed from the sample to yield a complex comprising the magnetic capture particle, the target nucleic acid and the target probe. The sample is contacted with a reporter particle comprising a plurality of binding groups, at least one of which is capable of binding with the target probe, wherein in the presence of the target nucleic acid, the reporter particles bind with target probes to provide a complex comprising the magnetic capture particle, the target nucleic acid, the target probe and the reporter particle. Unbound reporter particles are removed from the sample to provide a complex comprising the magnetic capture particle bound to the target nucleic acid bound to the target probe, which is bound to the reporter particle. The reporter particle is caused to disassociate from the target nucleic acid, and the presence of the reporter particle previously bound to the target nucleic acid is then detected.

In an embodiment of any of the above methods, the target probe binding group comprises an oligonucleotide capable of specifically binding to a binding site on a nucleic acid via a complementary nucleic acid base pairing interaction. For example, in some embodiments, a method comprises contacting a sample with a target probe and a capture particle (optionally magnetic), each comprising binding groups capable of specifically binding to one or more nucleic acid analytes by specific binding interactions. In some embodiments, a reporter particle including a plurality of binding groups capable of binding to the target probe in the presence of the analyte is contacted with the sample. Unbound reporter particle is separated from the sample. Unbound analyte can also be separated from analyte bound to the capture particle. The presence of the reporter particle in the sample is then detected. Optionally, reporter particle bound to the analyte by a target probe can be disassociated from the analyte prior to the detecting. For example, a target probe can be disassociated from an analyte-capture particle complex by disrupting a specific binding interaction between the target probe and the analyte and/or disrupting a specific binding interaction between the target probe and the reporter particle.

In one embodiment of any of the above methods, the method comprises contacting a disassociated reporter particle with a detector moiety prior to the detecting, wherein the detecting comprises measuring the value of a property of an agglomerate of the reporter particle and the detector moiety, wherein the value of a sample comprising the one or more analytes differs from the value of a reference sample lacking the one or more analytes.

In another embodiment of any of the above methods, detecting comprises determining a magnetic resonance relaxation time of a sample comprising one or more analytes compared to when a magnetic resonance relaxation time of a reference sample lacking the one or more analytes.

In still another embodiment of any of the above methods, a binding group present on a reporter particle, a target probe, a capture particle, and/or a detector moiety comprises an antibody capable of specifically binding to a site on an analyte selected from: a protein, a saccharide, an infectious agent, a cell, or a combination thereof.

In a particular embodiment of any of the above methods, (i) an analyte comprises a nucleic acid and (ii) a reporter particle, a target probe, a capture particle, and/or a detector moiety comprises an oligonucleotide capable of specifically binding to a nucleic acid sequence on the analyte via a specific nucleotide base-pairing interaction with the first nucleic acid sequence.

In certain embodiments of any of the above methods, the reporter particle comprises a plurality of biotin binding groups capable of binding to a target probe via a biotin-avidin interaction. For example, the detector moiety can comprise a plurality of avidin-functionalized binding groups capable of binding to a complexed or disassociated reporter particle via a biotin-avidin interaction.

For any of the above methods, the method can have a limit of detection of at least 1×10³, 1×10⁴, 1×10⁵, 1×10⁶, 1×10⁷, or 1×10⁸ analytes per milliliter of sample.

The present invention is also directed to a complex comprising an analyte, a magnetic capture particle comprising a first binding group bound to a first site on the analyte by a first specific binding interaction, a target probe comprising a second binding group bound to a second site on the analyte by a second specific binding interaction, wherein the first and second binding groups are different, and a reporter particle comprising a plurality of binding groups bound to a third binding group on the target probe, wherein the second and third binding groups are different.

In some embodiments, a magnetic capture particle present in a complex comprises a superparamagnetic particle having a cross-sectional dimension of 50 nm to 20 μm.

In some embodiments, a reporter particle present in a complex comprises a plurality of biotin binding groups and binds to a target probe via a biotin-avidin interaction.

In some embodiments, a complex comprises a nucleic acid analyte, the magnetic capture particle comprises a first oligonucleotide binding group bound to a first sequence of the nucleic acid analyte by a nucleotide base-pairing interaction, and the target probe comprises a second oligonucleotide binding group bound to a second sequence of the nucleic acid by nucleotide base-pairing interaction, wherein the first and second sequences of the nucleic acid are different.

The present invention is also directed to a reagent cartridge comprising a plurality of wells, each well suitable for holding a sealable container at a predetermined position, wherein the cartridge comprises a first sealable container at a first position that includes a reporter particle comprising a plurality of binding groups capable of binding to an analyte; and a second sealable container at a second position that includes detector moiety, wherein the detector moiety is magnetic, fluorescent, radioactive, or a combination thereof.

Additional features and advantages of the invention will be set forth in the description that follows, and in part will be apparent from the description, or can be learned by practice of the invention. The advantages of the invention will be realized and attained by the structure and particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIGS. 1-3 provide process flow diagrams for methods of the present invention.

FIGS. 4-5 provide cross-sectional schematic representations of complexes of the present invention.

FIGS. 6A-6B depict gel images resulting when non-covalent conjugates were electrophoresed on a native gel and stained with SYBR gold (FIG. 6A) (specific staining for nucleic acid) and Coomassie blue (FIG. 6B) (specific staining for streptavidin protein).

FIG. 7 provides a graphic representation of the change in T2 relaxation time plotted versus the density of DNA copies per mL of sample solution.

The present invention will now be described with reference to the accompanying drawings. In the drawings, like reference numbers indicate identical or functionally similar elements. Additionally, the left-most digit(s) of a reference number can identify the drawing in which the reference number first appears.

### DETAILED DESCRIPTION OF THE INVENTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

Throughout the specification, use of the term “about” with respect to any quantity is contemplated to include that quantity. For example, “about 10 μm” is contemplated herein to include “10 μm,” as well as values understood in the art to be approximately 10 μm with respect to the entity described.

References to spatial descriptions (e.g., “above,” “below,” “up,” “down,” “top,” “bottom,” etc.) made herein are for purposes of description and illustration only, and should be interpreted as non-limiting upon the methods, processes, articles, and products of any process of the present invention, which can be spatially arranged in any orientation or manner.

As used herein, “plurality” refers to 2 or more of an item, e.g., 2 or more, 5 or more, 10 or more, 50 or more, 100 or more, 1000 or more, etc., of an item.

The present invention is directed to methods of detecting one or more analytes in a sample. As used herein the term “sample” refers to a portion, piece, or segment that is representative of a whole. Sample for use with the present invention include liquids, solids, semi-solids (e.g., partially liquid samples, gels, sludge, and the like), aerosols, and combinations thereof. In some embodiments, a sample comprises one or more analytes, as well as non-analyte molecules, in a suitable volume or other configuration. Samples and one or more analytes for detection and measurement by the methods of the present invention can be of, e.g., biological and/or environmental origin. In some embodiments, a sample is of a bodily fluid (e.g., blood, urine, saliva, semen, serum, plasma CSF, feces, vaginal fluid or tissue, sputum, nasopharyngeal aspirate or swab, lacrimal fluid, mucous, epithelial swab (buccal swab) and the like) and thus is of biological origin from, e.g., a mammal such as a human. A sample can comprise biological materials from a subject such as, but not limited to, tissues, organs, bones, teeth, tumors, and the like. A sample can be a diluted sample comprising, e.g., a bodily fluid diluted with water or a suitable physiological buffer such as phosphate buffered saline, and the like. In some embodiments, a sample is a liquid sample to which one or more analytes and other components are added prior to detecting.

In some embodiments, a sample is held in a predetermined position by a device such as, but not limited to, an indentation, a vial, a well, a container, a tube, a recession, or other suitable element. A sample can be held in any suitable material such as, but not limited to, a polymer, a glass, a metal, a ceramic, and the like, and combinations thereof. In some embodiments, a sample is contained within a device that includes inert surfaces.

Analytes that can be detected by the methods of the present invention include, but are not limited to, proteins, nucleic acids, saccharides, lipids, small molecules, ions, gases, infectious agents, cells, and combinations thereof.

Proteins suitable for detection by the methods of the present invention include, but are not limited to, peptides, polypeptides, amino acids, glycoproteins, antibodies, antibody fragments, aptamers, and the like, and combinations thereof.

Nucleic acids suitable for detection by the methods of the present invention include, but are not limited to, siRNA, RNA, DNA, oligonucleotides thereof, synthetic variants thereof, and the like, and combinations thereof. As used herein, “target nucleic acid” refers to any length of DNA, RNA, or cDNA having any desirable sequence. As used herein, “oligonucleotides” refer to nucleic acids having lengths suitable to bind to a target nucleic acid. In some embodiments, an oligonucleotide complementary to a target nucleic acid is 3 base pairs to 100 base pairs in length, or 5 base pairs to 50 base pairs in length. As used herein, “complementary” refers to the interaction between a nucleic acid analyte and an oligonucleotide such that Watson-Crick base pairing occurs and hydrogen bonding results, thereby forming a target nucleic acid-complementary oligonucleotide structure. Construction of oligonucleotides complementary to a portion of the sequence of a target nucleic acid is performed using well known methods in the art.

Saccharides suitable for detection by the methods of the present invention include, but are not limited to, carbohydrates, disaccharides (e.g., sucrose, lactose, and the like), polysaccharides, proteoglycans, individual sugars (e.g., glucose, galactose, and the like), and combinations thereof.

Lipids suitable for detection by the methods of the present invention include, but are not limited to, lipoproetins, cholesterol, lipopolysaccharides, fatty acids, and the like, and combinations thereof.

Small molecules suitable for detection by the methods of the present invention include, but are not limited to, therapeutic compounds, diagnostic compounds, metabolites of therapeutic or diagnostic compounds, molecules used for research, and the like, and combinations thereof. As used herein, a “small molecule” is a therapeutic or diagnostic compound or a metabolite thereof having a molecular weight of 2,000 Da or less. In some embodiments, a small molecule has a molecular weight of 800 Da or less.

Gases suitable for detection by the methods of the present invention include, but are not limited to, gases found in organisms (either naturally or as a result of disease, disorder, and/or dysfunction, such as oxygen, oxygen radicals, carbon dioxide, hydrogen peroxide, and the like), and gaseous and/or aerosol warfare agents (e.g., cyanogen chloride, hydrogen cyanide, blister agents, ethyldichloroarsine, methyldichloroarsine, phenyldichloroarsine, lewisite, sulfur mustard gas, nitrogen mustard gas, tabun, sarin, soman, cyclosarin, EA-3148, VE, VG, VM, VR, VX, novichok agents, chlorine, chloropicrin, phosgene, diphosgene, agent 15, EA-3167, Kolokol-1, Pepper spray, CS gas, CN gas, and the like), and combinations thereof.

Ions suitable for detection by the methods of the present invention include, but are not limited to, electrolytes (e.g., sodium, potassium, calcium, ammonia, lactate, lactic acid, and the like), metals (e.g., transition metals such as iron, manganese, copper, chromium, zinc, and the like), and combinations thereof.

Cells suitable for detection by the methods of the present invention include, but are not limited to, viruses, bacteria, fungi, infective eukaryotic cells other than fungi, spores, and the like, and combinations thereof.

Infectious agents suitable for detection by the methods of the present invention include, but are not limited to, viruses, prions and prionic molecules, pathogens (e.g., anthrax, ebola, Marburg virus, plague, cholera, tularemia, brucellosis, Q fever, Bolivian hemorrhagic fever, coccidioides mycosis, glanders, nelioidosis, shigella, Rocky Mountain spotted fever, typhus, psittacosis, yellow fever, Japanese B encephalitis, rift valley fever, smallpox, and the like) naturally-occurring toxins (e.g., ricin, SEB, botulism toxin, saxitoxin, mycotoxins, and the like), and combinations thereof.

Furthermore, “detection of an analyte” can also refer to measurement of physical properties of a solution containing one or more analytes, for example, measurement of dipole moment, ionization, solubility/saturation, viscosity, gellation, crystallization, and/or phase changes of a solution.

In some embodiments, one or more analytes detected by the methods of the present invention are one or more biologically active substances and/or metabolite(s), marker(s), and/or other indicator(s) of biologically active substances. A “biologically active substance” can refer to a single entity, or a plurality of entities that are the same or different, and includes, without limitation: medications; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment. Examples of biologically active substances that can be detected using the methods described herein are disclosed in detail in, e.g., U.S. Pat. No. 7,564,245, the disclosure of which is incorporated by reference herein in its entirety for all purposes.

Further, one or more analytes detected by a method described herein can include drugs or medicaments that are being developed for therapeutic treatment of disease, disorders, or dysfunctions. The detection of the drug, medicament or metabolite in a pre-clinical development program can be useful for monitoring the concentration, levels, or bioavailability of the compound. Further, in a preclinical development program, detecting and/or monitoring the concentration, levels, or bioavailability can be correlated with the efficacy or toxic or adverse events. The detection of the drug, medicament or metabolite can further be useful for monitoring therapeutic effectiveness in a subject in a clinical trial, or in a patient after the drug or medicament has achieved marketing status. Rapid detection of active drug or medicament during a clinical trial can provide useful data and information to be included in a therapeutic product's marketing label. In addition, the specific drug or medicament that is under development can be analyzed in tandem with other biological features of the disease, disorder, or dysfunction, such as determining levels of a specific protein, nucleic acid, carbohydrate, lipid, ion, or cell and thus multiplexed detection of the drug, medicament or metabolite together with another biological determinant can optimize clinical decision making. Detection and/or monitoring of metabolites, can be particularly efficacious if a metabolite renders pharmacological activity similar to a parent drug, and can additionally be useful in clinical decision making. Detection and/or monitoring of a drug or metabolite may be useful to monitor unwanted toxic or adverse effects imparted by these compounds alone or together in a therapeutic regimen.

Further, many methods currently exist to correlate an individual's genotype or haplotype to therapeutic treatment options and therapeutic decision making. The general field of medical diagnostics has moved in the direction to fulfill the need to provide informed patient treatment options to clinicians. A subset of diagnostic tests that are specifically aimed at determining genotype and/or haplotype of the individual and then choosing the appropriate drug treatment regimen, timing, and monitoring effectiveness for an individual, subject, or patient based on the genetic make-up has been referred to broadly as personalized medicine. Diagnostics for personalized medicine are under development for use in settings where information is needed for a rapid decision (e.g. glucose testing and insulin adjustments; troponin testing and cardiac treatment) and in settings where information is not needed rapidly (e.g. cancer, neurological disorders, and immune based disease). Diagnostics for personalized medicine today is restrained by the capabilities of available tests. Currently, there is no available platform that can rapidly provide results multiplexed across target type boundaries (e.g. nucleic acid, protein, small molecule, infectious disease or agents), and currently there is no available platform that can provide results rapidly for target types that necessitate sample preparation (e.g. extraction, purification, etc). Thus, personalized medicine is currently constrained to conditions where sample requirements with respect to turnaround time, sample purification/matrix type, and multiple analyte capability are not factors in the linkage of disease state to therapy. The present method provides a solution to these constraints.

The methods and devices of the invention may be used to detect a very wide range of biologically active substances, as well as other analytes. Of current methods (e.g. chemiluminescence, nephelometry, photometry, and/or other optical/spectroscopic methods), no single approach can achieve the diversity of analysis that is possible with NMR, even without the sensitivity improvements made possible by embodiments described herein. The sensitivity improvements provided by embodiments of the invention described herein allow further breadth and adaptability of analysis over current NMR techniques. For example, embodiments of the invention may be used or adapted for detection, for example, of any protein (e.g., biomarkers for cancer, serum proteins, cell surface proteins, protein fragments, modified proteins), any infectious disease (e.g., bacterial based on surface or secreted molecules, virus based on core nucleic acids, cell surface modifications, and the like), as well as a wide range of gases and/or small molecules.

A wider range of drugs may be developed, due to the improved ability to detect and maintain appropriate dosages using the NMR devices and methods described herein. Drugs may be administered either manually or automatically (e.g., via automatic drug metering equipment), and may be monitored intermittently or continuously using the device. Dosage may therefore be more accurately controlled, and drugs may be more accurately maintained within therapeutic ranges, avoiding toxic concentrations in the body. Thus, drugs whose toxicity currently prevents their use may become approved for therapeutic use when monitored with the device or method described herein.

Medical conditions that may be rapidly diagnosed by the method for proper triaging and/or treatment include, for example, pain, fever, infection, cardiac conditions (e.g., stroke, thrombosis, and/or heart attack), gastrointestinal disorders, renal and urinary tract disorders, skin disorders, blood disorders, and/or cancers. Tests for infectious disease and cancer biomarkers for diseases not yet diagnosable by current tests may be developed and performed using the NMR device or method described herein.

The device or method may be used for detection of chemical and/or biological weapons in the field, for example, nerve agents, blood agents, blister agents, plumonary agents, incapacitating agents (e.g., lachrymatory agents), anthrax, ebola, bubonic plague, cholera, tularemia, brucellosis, Q fever, typhus, encephalitis, smallpox, ricin, SEB, botulism toxin, saxitoxin, mycotoxin, and/or other toxins.

Because the devices and methods are adaptable for detection of multiple analytes, a unit may be used to perform many ICU tests (including, e.g., PICU, SICU, NICU, CCU, and PACU) quickly and with a single blood draw. The tests may also be performed in the emergency room, in the physician's office, in field medicine (e.g., ambulances, military medical units, and the like), in the home, on the hospital floor, and/or in clinical labs. The multiplexing capability of the devices also makes them a valuable tool in the drug discovery process, for example, by performing target validation diagnostics.

Measurements for one or more analytes may be made, for example, based on a single draw, temporary draws, an intermittent feed, a semi-continuous feed, a continuous feed, serial exposures, and/or continuous exposures. Measurements may include a detection of the presence of the one or more analytes and/or a measurement of the concentration of one or more analytes present in the sample.

As used herein, “contacting” or “contacted” refers to the introduction, mixing or placement of components together so that the components interact with one another. Contacting includes, but is not limited to, mixing two liquids with each other, adding a liquid to a solid, paste, gel, and/or particulate, adding a gas, solid, paste, gel, and/or particulate to a liquid, and the like, and combinations thereof.

In some embodiments, the methods of the present invention include contacting a sample with a reporter particle. As used herein “reporter particle” refers to a molecule, moiety, species, and the like that can aid in the detection of one or more analytes. A reporter particle suitably comprises a plurality of binding groups capable of binding to an analyte, a target probe, a capture particle, and/or a detector moiety.

As described herein, a reporter particle suitably does not comprise a detection moiety. That is, a reporter particle does not include a fluorescent moiety, molecule, species, tag, and/or label, a radioactive moiety, tag, species, and/or label, or another detection marker. Thus, the reporter particles aid in the detection of one or more analytes by enhancing or otherwise facilitating agglomeration of reporter particles with detector moieties, capture particles, and/or analytes, but reporter particles themselves are not required to be detected or detectable.

In some embodiments, a reporter particle has a cross-sectional dimension of 50 nm to 10 μm, or 100 nm to 7.5 μm, or 500 nm to 5 p.m.

In some embodiments, a reporter particle is free from a magnetic element or compound. That is, reporter particles are not influenced by the application of a magnetic field.

A reporter particle is suitably a polymeric particle (although materials including, but not limited to, metals, metal oxides, ceramics, biopolymers, biomolecules, and the like, can also be used) comprising a plurality of binding groups moieties. In exemplary embodiments, reporter particles comprise a polymer such as polystyrene, and have a cross-sectional dimension of 800 nm to 3 μm, or about 1 μm. Exemplary polymeric particles for use with the present invention include POLYBEAD® microspheres, POLYBEAD® functionalized microspheres (POLYSCIENCES, INC.®, SA.), and the like.

A reporter is capable of binding to one or more analytes. As used herein, “binding” refers to two or more species interacting in a physiochemical manner proximate one another such that energy (i.e., the binding energy) is required to separate the species from one another. Binding interactions suitable for use with the present invention include both specific and non-specific binding interactions such as, but not limited to, hydrogen bonding, a hybridization interaction, pi-pi stacking, metal-organic binding, protein-substrate binding, antibody-antigen binding, covalent bonding, ionic bonding, and the like, and combinations thereof.

Binding groups suitable for use with the present invention include, but are not limited to, nucleic acids (e.g., oligonucleotides), polypeptides (e.g., proteins), antibodies, saccharides (e.g., polysaccharides), lipids, small molecules, and the like. In some embodiments, a binding group is a synthetic oligonucleotide that hybridizes with a specific complementary nucleic acid target. In some embodiments, a binding group is an antibody directed toward an antigen or protein involved in a protein-protein interaction. In some embodiments, a binding group is a polysaccharide that binds to a corresponding target or protein, such as avidin or biotin. Examples of suitable binding groups are also described throughout U.S. Pat. No. 7,564,245, the disclosure of which is incorporated by reference herein in its entirety for all purposes.

In some embodiments, a reporter particle comprises a plurality of biotin binding groups capable of binding to a target probe via a biotin-avidin interaction.

In some embodiments, at least two or more of binding groups are accessible such that the two or more binding groups can simultaneously hybridize or bind to a corresponding or complementary binding partner or target molecule.

Binding groups can be attached directly to a surface of a reporter particle or can be attached to a reporter particle via a linker or spacer. Linker and spacer groups suitable for use with the present invention are not particularly limited, and include those linker and spacer groups known in the biological, chemical, and biochemical arts.

In some embodiments, the methods of the present invention include contacting a sample with a target probe. As used herein, a “target probe” refers to a moiety that binds specifically to two or more different species. For example, a target probe can bind to an analyte and a reporter particle, an analyte and a detector moiety, a reporter particle and a detector moiety, an analyte and a capture particle, and/or a capture particle and a reporter particle. In some embodiments, a target probe comprises at least one functional group that binds to an analyte, and further functional groups suitable for binding with a reporter particle, a detector moiety, a capture particle, or a combination thereof. In some embodiments, a target probe comprises three or more binding groups.

In some embodiments, a target probe comprises a particle. Particles suitable for use as a portion of a target probe include those particles described herein as suitable for use as reporter particles, supra. In some embodiments, a target probe comprises a particle that includes at least two different binding groups on the surface of the particle, for example, an oligonucleotide and a biotin binding group.

A target probe can comprise a single molecule or a complex/multiplex comprising two or more distinct molecules. Exemplary target probes include oligonucleotides comprising a nucleic acid sequence complementary to a target nucleic acid sequence of an analyte, further functionalized with one or more binding groups (e.g., a small molecule, a protein, a nucleic acid, an antibody, a virus, a biotin, an avidin, and the like, and combinations thereof). In some embodiments, a target probe comprises one or more streptavidin or biotin binding groups.

In some embodiments, a target probed comprises a first binding group capable of binding to a first target site on an analyte and a second binding group capable of binding to a reporter particle or a detector moiety, wherein in the presence of one or more analytes, the target probe binds to the first target site on the analyte via the first binding group and binds to the non-magnetic reporter particle or the detector moiety via the second binding group.

In some embodiments, a sample comprising one or more analytes is contacted with a target probe capable of binding to the one or more analytes, and the sample is also contacted with a reporter particle capable of binding to the target probe.

In some embodiments, a sample comprising one or more nucleic acid analytes is contacted with a target probe comprising an oligonucleotide capable of specifically binding to a binding site on the target nucleic acid via a complementary nucleotide base pairing interaction.

In some embodiments, a sample comprising one or more protein, saccharide, infectious agent, and/or cell analytes is contacted with a target probe comprising an antibody binding group capable of specifically binding to a first target site on the protein, saccharide, infectious agent, and/or cell. Subsequently, the sample is contacted with a reporter particle and/or capture particle capable of binding with a second binding group on the target probe. In some embodiments, the second binding group on the target probe is a biotin suitable for binding with an avidin protein.

In some embodiments, the methods of the present invention include contacting a sample with a detector moiety. As used herein, a “detector moiety” refers to a species capable of binding to one or more analytes, reporter particles, target probes, capture particles, other detector moieties, or combinations thereof (e.g., binding to both a reporter particle and an analyte) to form an agglomerate. A detector moiety comprises a species, tag, label, molecule, particle, and the like capable of being detected using one or more analytical methods. In some embodiments, a detector moiety includes a species such as, but not limited to, a magnetic particle, a fluorescent moiety (e.g., a fluorescent molecule, tag, label, and/or particle, e.g., FLUORESBRITE® particles (POLYSCIENCES, INC.®, SA.), and the like), a radioactive moiety (e.g., a radioactive molecule, tag, label, and the like), a chiral molecule, UV-absorbing species, and visible-absorbing species (e.g., POLYBEAD® dyed microspheres, POLYBEAD® carboxylate dyed microspheres (POLYSCIENCES, INC.®, SA), and the like), and combinations thereof. Exemplary fluorescent moieties are well known in the art and include fluorescein, rhodamine, Alexa Fluors, Dylight fluors, ATTO Dyes, as well as others, and can be purchased e.g., from Molecular Probes, Eugene Oreg. Exemplary radioactive moieties include tritium (³H), ¹⁴C, ³⁵S, ²²S, ¹³⁶C, ³²P, ¹²⁵I, and ³³P as well as others that are well known in the art. Chiral molecules are well known in the art, and include any molecule having a center of chirality. UV-absorbing and visible-absorbing species are also well known in the art, and include any species having an extinction coefficient at a wavelength of 200 nm to 700 nm of about 5,000 L mol⁻¹ cm⁻¹ or greater, or about 10,000 L mol⁻¹ cm⁻¹ or greater.

In some embodiments, a detector moiety comprises a magnetic particle. Magnetic particles suitable for use with the present invention include superparamagnetic iron oxide (SPIO) particles, including functionalized SPIO particles such as avidinated or biotinylated SPIO particles. In some embodiments, magnetic particles have a cross-sectional dimension of 50 nm to 20 μm, 100 nm to 15 μm, 500 nm to 5 μm, 750 nm to 1 μm, about 1 μm, or about 2 μm. Magnetic particles suitable for use with the present invention further include, but are not limited to, DYNABEADS® MYONE™ Streptavidin-C 1 coated superparamagnetic particles (INVITROGEN DYNAL® AS, Oslo, Norway), and PROMAG™, BIOMAG®, and BIOMAG® Plus particles (POLYSCIENCES, INC.®, SA.), and the like. Additional magnetic particles suitable for use with the present invention are disclosed in, U.S. Pat. Nos. 4,554,088, 5,055,288, 5,262,176, 5,512,439, and 7,459,145, and U.S. Pub. Nos. 2003/0092029, 2003/0124194, 2006/0269965, and 2008/0305048, which are incorporated herein by reference in the entirety.

In some embodiments, a detector moiety comprises a plurality of avidin-functionalized binding groups capable of binding to a reporter particle via a biotin-avidin interaction. As discussed herein, the reporter particle can be bound to an analyte or an analyte-target probe complex during the binding with a detector moiety. Alternatively, the reporter particle is disassociated from an analyte or a target probe and then contacted with a detector moiety.

In some embodiments, a sample is contacted with a plurality of magnetic detector moieties. As described herein, contacting a sample with a plurality of magnetic detector moieties can lead to a variety of different binding interactions. The detector moieties can bind to the reporter particle, one or more analytes, or a combination thereof. Binding can occur directly between multiple binding groups on the surface of a reporter particle and the magnetic detector moieties. In other embodiments, as described herein, functionalized magnetic particles can be utilized that bind to the binding groups on the surface of the reporter particle. The reporter particles aid in the agglomeration of the magnetic detection particles. This agglomeration can be detected via various methods, including magnetic resonance (such as the measurement of a relaxation parameter) or use of optical or other methods to detect the agglomeration. For example, in the presence of one or more analytes, a reporter particle can enhance agglomeration of magnetic detector moieties, which results in an analyte being detected by a change in a property of a sample when one or more analytes are present in a sample compared to a sample lacking the one or more analytes. In embodiments in which the detector moiety comprises a magnetic particle, the property of the sample can include a relaxation time measurable by NMR spectroscopy. Exemplary analytes that can be detected using the methods of the present invention are described throughout, and suitably include a protein, a nucleic acid, a saccharide, a lipid, a small molecule, an ion, a gas, an infectious agent, a cell, and combinations thereof.

In suitable embodiments, contacting a sample with a reporter particle occurs prior to contacting a sample with a detector moiety. Furthermore, removing unbound reporter particles from a sample can occur anytime after contacting a sample with a reporter particle. That is, unbound reporter particle can be removed before or after the addition of detector moieties. In some embodiments, contacting the sample with a reporter particle occurs prior to contacting a sample with a detector moiety, and unbound reporter particles are removed from a sample after contacting with the detector moiety. Additionally, in some embodiments unbound reporter particles are not removed from a sample at any point (i.e., in some embodiments unbound reporter particles can remain in the sample during the detecting).

Removing unbound reporter particles from a sample can comprise washing a sample with a solvent or diluent (e.g., water, saline, and the like) to remove reporter particles that are not bound to an analyte. Suitable washing methods are known in the art and include, for example, various centrifugation, vortexing or mixing, and dilution/elution steps. Removing unbound reporter particles from a sample can also include filtering, chromatography, and the like.

In some embodiments, reporter particles are disassociated from the analyte after the removing and prior to the detecting. Reporter particles can be disassociated from an analyte by a process comprising temperature denaturing, generating a pH gradient, reducing disulfide bonds, oxidizing disulfide bonds, mechanically disrupting, or a combination thereof, so as to disrupt the interaction between an analyte and a reporter particle, a target probe and an analyte, and/or a target probe and a reporter particle. For example, a target probe can be disassociated from an analyte by disrupting a specific binding interaction between the first binding group on the target probe and the first binding site on the analyte.

The methods of the present invention comprise detecting an agglomerate. As used herein, “agglomeration” refers to a process of clustering, agglutination and/or coming together of various species to form an agglomerate, cluster, aggregate, and the like. Agglomeration can occur via various mechanisms. For example, a reporter particle can enhance agglomeration of a plurality of detector moieties, for example, by binding to multiple analytes and/or detector moieties, thus bringing these species into proximity with one another and assisting with the formation of an agglomerate.

In some embodiments, a sample is subjected to magnetic assisted agglomeration prior to the detecting. Magnetic assisted agglomeration can assist in the formation of agglomerates/clusters/aggregates of magnetic particles (e.g., an agglomerate comprising a detector moiety comprising a magnetic particle and a magnetic capture particle, and optionally, an analyte, if still present in the sample). Exemplary methods for carrying out magnetic assisted agglomeration are described herein as well as in Koh et al., “Sensitive NMR Sensors Detect Antibodies to Influenza,” Angew. Chem. Int. Ed. 47:1-4 (2008), the disclosure of which is incorporated by reference herein in its entirety for all purposes. As discussed in Koh et al., agglomeration of magnetic particles prior to detection can be enhanced by the application of a homogeneous (i.e., a non-varying force throughout the sample) magnetic field, followed by removal of the magnetic field to allow for any deaggregation to occur.

Agglomeration can be detected by various methods and devices, including magnetic resonance methods, fluorescence detection methods, optical detection methods, changes in electrical properties of a sample, changes in density, mass, turbidity, and/or rheological properties of the sample, and the like. Exemplary methods of detecting agglomeration in a sample include, but are not limited to, determining a magnetic resonance property of a sample, determining a relaxation time (including T1, T2 and/or T2* times) of a sample, determining the turbidity of a sample, determining the density of a sample, determining the rheology of a sample, measuring the circular dichroism of a sample, measuring the ultraviolet and/or visible absorption spectrum of a sample, and/or measuring the radioactivity of a sample. Methods of making such measurements/determinations and devices for carrying out these methods are well known in the art. Exemplary methods and devices for determining a relaxation time of a sample can be found throughout, e.g., U.S. Pat. No. 7,564,245, the disclosure of which is incorporated by reference herein in its entirety for all purposes.

Agglomeration or aggregation within a sample can be detected by any method or device that determines an enhancement, augmentation, change or response in agglomeration in a composite, as compared to a sample containing un-agglomerated or less agglomerated species (e.g., a sample containing only one or more analytes and reporter particles (i.e., lacking detector moieties), a sample containing only one or more analytes and detector moieties (i.e., lacking reporter particles and/or capture particles), a sample containing only reporter particles and detector moieties (i.e., lacking one or more analytes), etc.

Not being bound by any particular theory, reporter particles can facilitate or assist in agglomeration of magnetic capture particles. Such agglomeration can be detected by various methods described herein, including magnetic resonance spectroscopy (e.g., the measurement of a relaxation parameter), optical methods, or other analytical methods known to persons of ordinary skill in the art.

In some embodiments, the presence of an analyte in an aqueous sample provides either an increase or a decrease in T2 relaxation time compared to an aqueous sample lacking the analyte. The change in T2 relaxation time (i.e., the increase or decrease in T2 relaxation time) can be correlated with the concentration of the analyte in the sample, thereby providing a quantitative measurement of the analyte's presence in the sample.

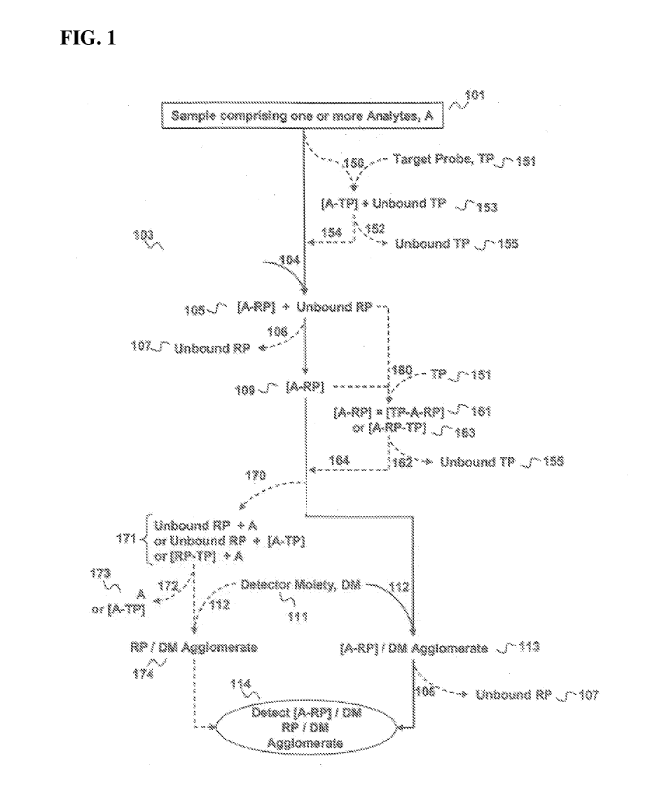

FIG. 1 provides a schematic flow-chart illustrating various embodiments of the present invention. Referring to FIG. 1, a sample, 101, comprising one or more analytes (A) is contacted, 104, with a reporter particle, 103 (RP), capable of binding to the one or more analytes, wherein in the presence of an analyte, the reporter particle binds to the analyte to form an analyte-reporter particle complex, 105 [A-RP]. Also present in the sample is unbound reporter particle, 107 [RP], which is not bound to an analyte.

In some embodiments, the methods of the present invention comprise separating species that do not undergo a specific binding and/or agglomeration interaction from a sample. For example, an unbound analyte, an unbound reporter particle, an unbound target probe, an unbound capture particle, and/or an unbound detector moiety, can be separated from a sample comprising a complex. Separating can be performed, for example, by applying a magnetic field to a sample, filtering the sample, chromatographically treating the sample, and the like. In some embodiments, separating can enhance the detecting, for example, yielding a more accurate measurement of a magnetic resonance property (e.g., T2 relaxation time). Referring to FIG. 1, in some embodiments the unbound reporter particle 107, is optionally removed, 106, from the sample.

Referring to FIG. 1, the sample, 101, can be optionally contacted, 150, with a target probe, 151 (TP). In the presence of an analyte, a composition, 153, is provided comprising an analyte-target probe complex [A-TP] and unbound target probe. The unbound target probe, 155, can be optionally removed, 152, from the sample, and the process can be resumed, 154, as described above. However, instead the sample is contacted with a reporter particle capable of binding to the target probe or the analyte. Thus, a reporter particle can binding directly to an analyte, or bind to an analyte via the target probe (thereby forming an [A-TP-RP] complex). Suitably, as described herein, the reporter particle comprises a plurality of binding groups (i.e., the reporter particle is multivalent).

Referring to FIG. 1, the sample comprising the [A-RP] complex, 109, is contacted, 112, with a detector moiety, 111 (DM), to provide an agglomerate, 113, comprising the analyte-reporter particle complex, [A-RP], and a plurality of detector moieties. If present in the sample, 113, unbound reporter particle, 107, can be optionally removed, 106, from the sample after contacting with the detector moiety. Thus, the unbound reporter particle can be optionally removed, 106, prior to contacting with a detector moiety, or after contacting with a detector moiety. Alternatively, unbound reporter particle can remain in the sample.

Referring to FIG. 1, a property of the sample comprising the analyte-reporter particle/detector moiety agglomerate, 113, is then detected, 114, by methods described herein. Not being bound by any particular theory, agglomeration of the reporter particle and detector moiety in the presence of one or more analytes is compared with a property of a reference sample lacking one or more analytes.

Referring to FIG. 1, a sample comprising the analyte-reporter particle complex, 109, is optionally contacted, 160, with a target probe, 151. Such contacting, 160, can occur after contacting, 104, with a reporter particle and prior to contacting, 112, with a detector moiety. The sample, 109, comprising the [A-RP] complex (with or without unbound reporter particle, 107) is optionally contacted, 160, with a target probe, 151, wherein the target probe binds to the analyte or the reporter particle to provide a target probe-analyte-reporter particle complex, 161 [TP-A-RP], or an analyte-reporter particle-target probe complex, 163 [A-RP-TP]. Unbound target probe, 155, is optionally removed, 162, from the sample, and the process is resumed, 164, as described above except that the [TP-A-RP] complex, 161, or [A-RP-TP] complex, 163, agglomerates with the detector moiety.

The methods can optionally comprise separating an unbound reporter particle from the sample, and then disassociating a bound reporter particle from the analyte. Thus, in addition to the methods just described, prior to the detecting, a reporter particle bound to an analyte and/or a target probe is optionally disassociated from a complex with an analyte. Referring to FIG. 1, a composition, 109, comprising the [A-RP] complex from which unbound reporter particle, 107, has been removed, 106, is subjected to conditions under which the reporter particle disassociates, 170, from the analyte to provide a composition, 171, comprising at least one of: unbound reporter particle and unbound analyte, unbound reporter particle and target probe-labeled analyte, or unbound analyte and target probe-labeled reporter particle. Specifically, the disassociating, 170, can affect any of an analyte-reporter particle binding interaction, an analyte-target probe binding interaction, or a reporter particle-target probe binding interaction. The unbound analyte or [A-TP] complex, 173, can be optionally separated, 172, from the sample (e.g., using an affinity column, resin, and the like) to provide a composition comprising unbound reporter particle (optionally labeled with a target probe).

In some embodiments, disassociated reporter particles are contacted with detector moieties (e.g., magnetic detector moieties) capable of binding to the unbound reporter particle. The unbound reporter particles can enhance agglomeration of the magnetic detector moieties. Referring to FIG. 1, a detector moiety, 111, is optionally contacted, 112, with the sample comprising the unbound reporter particles, wherein the reporter particles and detector moieties form a reporter particle-detector moiety agglomerate, 174 (RP/DM), that can be detected, 114, using methods described herein.

In some embodiments, the present invention is directed to a process for detecting a target nucleic acid in a sample, the method comprising contacting a sample comprising one or more nucleic acid analytes with a reporter particle comprising a plurality of oligonucleotides attached thereto. In the presence of a nucleic acid analyte having a base-pair sequence complementary to the sequence of the oligonucleotide a complex is formed between a nucleic acid analyte and a reporter particle. Unbound reporter particle is then removed from the sample and/or the complexes are removed from the sample. The sample comprising the complexes is then contacted with a detector moiety, wherein in the presence of the reporter particle bound to the analyte, the reporter particle facilitates aggregation of the detector moieties. Alternatively, the reporter particles can be disassociated from the analytes, optionally isolated, and then contacted with the detector moieties. The presence of the analyte is then detected by determining a property of the sample corresponding to the degree of aggregation within the sample. For example, the T2 relaxation time of the sample can be measured by methods described herein, wherein the T2 relaxation time of the sample comprising the target nucleic acid analyte will be increased or decreased compared to a sample lacking the target nucleic acid.

The present invention is also directed to a method of detecting one or more analytes in a sample, the method comprising contacting the sample with a capture particle comprising a first binding group capable of specifically binding to a first binding site on the one or more analytes, wherein in the presence of an analyte, the capture particle binds to the first binding site; contacting the sample with a reporter particle comprising a plurality of binding groups capable of binding to the analyte-capture particle complex, wherein in the presence of the analyte, the reporter particle binds to the analyte-capture particle complex; removing unbound reporter particle from the sample; and detecting the presence of the reporter particle.

Thus, in some embodiments, the methods of the present invention comprise contacting a sample with a capture particle capable of binding to a first target site on one or more analytes. As used herein, a “capture particle” refers to a particle comprising a binding group capable of specifically binding to an analyte to form an analyte-capture particle complex, wherein the capture particle has a property, binding group, functional group, and the like, sufficient for isolating the capture particle from a sample. For example, capture particles can include a second binding group (e.g., —NH₂ group, —NH₃⁺ group, —COOH group, —COO⁻ group, −SH group, and the like) suitable for reversible immobilization on a membrane, packed column, a metal surface, and the like. In some embodiments, a capture particle comprises a magnetic portion, thereby enabling magnetic-assisted separation/isolation of a capture particle-analyte complex from/within a sample.

As used herein a “magnetic capture particle” refers to a particle comprising a plurality of binding groups and having a magnetic portion (e.g., a core, shell, or combination thereof). Materials suitable for use in magnetic capture particles with the present invention include, but are not limited to, iron, iron oxide, nickel, cobalt, gadolinium, and alloys thereof. Binding groups include those described elsewhere herein, e.g., a protein, an antibody, a nucleic acid, and/or a small molecule, which is directly bound to a surface of the magnetic capture particle and/or attached to a non-magnetic portion of a particle. Attachment can be direct or include optional linkers and/or spacers. Various chemical linkers useful to attaching magnetic particles to binding groups are known in the art. In some embodiments, magnetic capture particles are functionalized with carboxylate (—COO⁻) groups. In some embodiments, a capture particle comprises a plurality of binding groups thereon such that multiple analytes can be bound to a single capture particle. In the embodiments whereby a magnetic capture particle is employed to separate a formed complex from unbound particles or assay components, magnetic capture particles may also require removal to limit interference of magnetic capture particles with magnetic detector particles.

In some embodiments, a sample comprising one or more analytes is contacted with a capture particle that includes a first binding group capable of specifically binding to a target site on the one or more analytes to form an analyte-capture particle complex. The sample is then contacted with a target probe that includes a second binding group capable of binding with a second target site on the one or more analytes or binding with a second binding group on the capture particle. Thus, a [A-CP-TP] or [TP-A-CP] complex is formed. The sample is then contacted with a reporter particle capable of binding to a second binding group on the target probe. Typically, the first and second binding groups on the target probe and the first and second binding groups on the capture particle are each unique (and differ from one another).

In some embodiments, reporter particles are disassociated from a complex comprising an analyte prior to detecting. In such embodiments, even though the analyte is removed prior to detecting, the presence of reporter particles is nonetheless a direct measure of the presence of the analyte in a sample. As discussed herein, suitable disassociating methods include, but are not limited to, temperature denaturing, generating a pH gradient, reducing disulfide bonds, oxidizing disulfide bonds, mechanically disrupting, or other suitable method, or combinations thereof. Disassociation of the reporter particle from a complex comprising an analyte can involve breaking or disrupting bonds or associations between the reporter particle and analyte, e.g., at a target site on the analyte to which the reporter particle or target probe is bound. In embodiments where a target probe is utilized, this removal can occur by disrupting the interaction between the first target site and the first target molecule (e.g., by disrupting a protein-protein interaction or a nucleic acid-nucleic acid base pair interaction).

As described herein, in some embodiments detecting an analyte comprises detecting the presence of the reporter particle. For example, in embodiments utilizing a capture particle, prior to the detecting a reporter particle can be optionally disassociated from a complex comprising an analyte. Therefore, if no analyte is present to bind with a reporter particle there will be a significantly lower concentration of the reporter particle upon disassociation from the analyte. Thus, the presence of a reporter particle during the detecting is indicative of the presence of previous binding between an analyte and reporter particle. In this manner, the reporter particle amplifies the presence of an analyte in a sample without requiring enzymatic duplication, and the like, of an analyte.

In some embodiments, the presence of a reporter particle is verified by measuring a property of the sample corresponding to agglomeration of the reporter particle. Optionally, a reporter particle is contacted with a detector moiety prior to and/or during the detecting, and an agglomerate comprising the reporter particle and detector moiety is thereby formed. The properties of the reporter particle-detector moiety agglomerate can be detected by the methods described herein, and include determining a change in T2 relaxation time of the sample as a result of the agglomeration of magnetic particles. Exemplary methods for determining a change in T2 relaxation time are known in the art and described, for example, throughout U.S. Pat. No. 7,564,245, the disclosure of which is incorporated by reference herein in its entirety for all purposes.

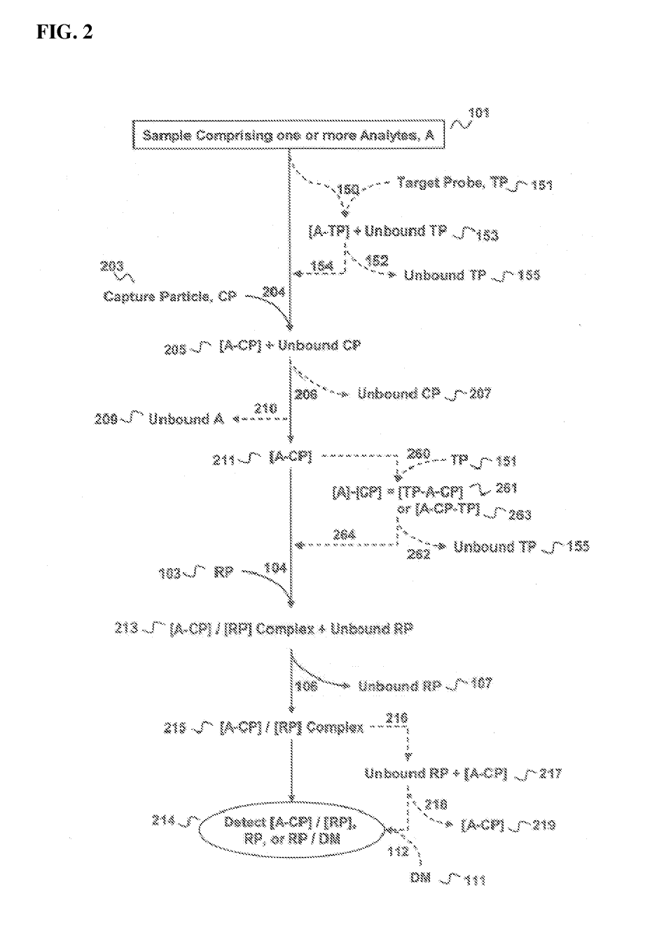

FIG. 2 provides a schematic flow-chart illustrating various embodiments of the present invention. Referring to FIG. 2, a sample, 101, comprising one or more analytes (A) is contacted, 204, with a capture particle, 203 (CP), capable of specifically binding to a first binding site on the one or more analytes. When an analyte is present in the sample, contacting the capture particle, 203, and the sample, 101, provides a composition, 205, comprising an analyte-capture particle complex [A-CP] with unbound capture particle. Specifically, the capture particle binds to a first binding site on an analyte. The unbound capture particle, 207, is then removed, 206, from the sample, to provide a sample comprising an analyte-capture particle complex, 211. In addition to separating, 206, unbound capture particle, 207, from the sample, unbound analyte, 209, can also be optionally separated, 210, from the sample. In some embodiments, the separating comprises isolating the [A-CP] complex, 211, from the sample, for example, using a magnetic field, column chromatography, a resin, a filter, centrifugation, and the like, and combinations thereof.

Referring to FIG. 2, the sample, 101, can be optionally contacted, 150, with a target probe, 151 (TP). In the presence of an analyte, a composition, 153, is provided comprising an analyte-target probe complex [A-TP] and unbound target probe. The unbound target probe, 155, can be optionally removed, 152, from the sample, and the process can be resumed, 154, as described above, except that a capture particle can bind to either a first binding site on an analyte (to form a target probe-analyte-capture particle complex, [TP-A-CP] (261) or a binding site on the target probe (to form an analyte-target probe-capture particle complex, [A-TP-CP] (263).

Referring to FIG. 2, the sample, 101, can be optionally contacted, 150, with a target probe, 151 (TP). For example, a target probe, 151 (TP) is optionally added prior to contacting the sample with a capture particle, 203, such that an analyte-target probe complex, 153 [A-TP], is formed. Unbound target probe, 155, is then removed, 152, from the sample, and the process can be resumed, 154, as described above except that instead of binding directly to an analyte, a capture particle can bind with an analyte via the target probe (thereby forming an [A-TP-CP] complex (263).

Referring to FIG. 2, the resulting sample comprising the [A-CP] complex, 211, is contacted, 104, with a reporter particle, 103 (RP), to provide a composition, 213, comprising an analyte-capture particle/reporter particle complex along with unbound reporter particle. Binding between the reporter particle, 103 (RP), and the [A-CP] complex can occur via the analyte or the capture particle. In some embodiments, the reporter particle, 103 (RP), binds to the [A-CP] complex, 211, via a specific binding interaction between the reporter particle and the analyte. For example, a binding moiety present on the reporter particle binds specifically with a second binding site on the analyte. Unbound reporter particle, 107 [RP], is then removed, 106, from the sample.

Referring to FIG. 2, a composition, 215, comprising an [A-CP]/[RP] complex is then detected, 214, by methods described herein.

Referring to FIG. 2, prior to contacting with a reporter particle, a composition, 211, comprising an analyte-capture particle complex [A-CP] is optionally contacted, 260, with a target probe, 151. Such contacting can occur after contacting, 204, with a capture particle and prior to contacting, 104, with a reporter particle. The sample, 211, comprising the [A-CP] complex (from which unbound CP has been removed, 206) is optionally contacted, 260, with a target probe, 151, wherein the target probe binds to the analyte or the capture particle to provide a target probe-analyte-capture particle complex, 261 [TP-A-CP], or an analyte-capture particle-target probe complex, 263 [A-CP-TP]. Unbound target probe, 155, is optionally removed, 262, from the sample, and the process is resumed, 264, as described above except that the [TP-A-CP] complex, 261, or [A-CP-TP] complex, 263, is then contacted with a reporter particle.

Referring to FIG. 2, after contacting, 104, with a reporter particle, 103, and also removing, 106, unbound reporter particle, 107, a sample comprising an [A-CP]/[RP] complex, is optionally treated, 216, to disassociate the reporter particle from the complex. Thus, in some embodiments a method comprises disassociating a bound reporter particle from the analyte prior to the detecting. The disassociating can comprise, for example, releasing the reporter particle from the analyte-capture particle complex by disrupting a specific binding interaction between: the reporter particle and the analyte, the reporter particle and the capture particle, the reporter particle and a target probe, target probe and the capture particle, or a target probe and the analyte. Suitable disassociating processes include those described herein elsewhere. The resulting composition, 217, comprises unbound reporter particle and an analyte-capture particle complex [A-CP]. The [A-CP] complex, 219, is then separated, 218, from the unbound reporter particle, 218, and the reporter particle is detected, 214, by methods described herein. Alternatively, prior to the detecting, the disassociated reporter particle is optionally contacted, 112, with a detector moiety, 111, to provide a reporter particle-detector moiety agglomerate, RP/DM. In such cases, the detecting, 114, comprises measuring a property of the sample corresponding to agglomeration of the reporter particle and the detector moiety, wherein the property of a sample comprising the one or more analytes differs from the property of a reference sample lacking the one or more analytes.

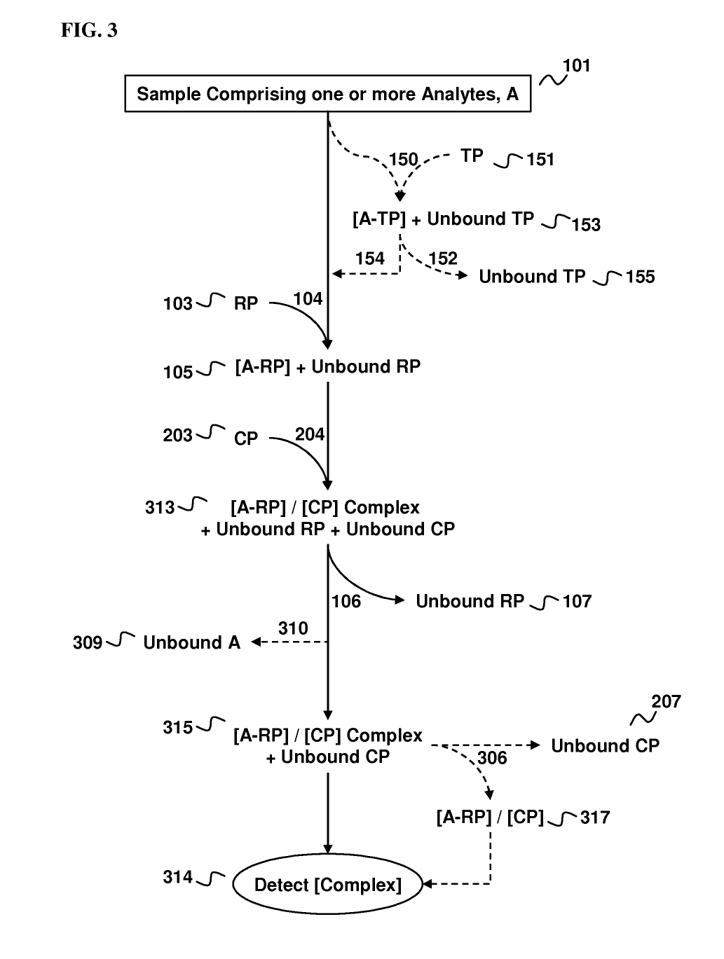

The order of the steps is not critical to the invention. FIG. 3 provides an additional schematic flow-chart illustrating various embodiments of the present invention. Referring to FIG. 3, a sample, 101, comprising one or more analytes (A) is contacted, 104, with a reporter particle, 103 (RP), capable of binding to the one or more analytes. When an analyte is present in the sample, the reporter particle, 103, forms a complex with the one or more analytes, thereby providing a composition, 105, comprising an analyte-reporter particle complex, [A-RP], and unbound reporter particle. The composition is then contacted, 204, with a capture particle, 203. In the presence of an analyte, the capture particle binds to the [A-RP] complex via a specific binding interaction with either the analyte (to form a capture particle-analyte-reporter particle complex, [CP-A-RP]) and/or a specific binding interaction with the reporter particle (to form an analyte-reporter particle-capture particle complex, [A-RP-CP]). Thus, in the presence of an analyte contacting the sample with a capture particle provides a composition, 313, comprising an analyte-reporter particle complexed with a capture particle. Optionally present in the composition, 313, is unbound reporter particle and unbound capture particle. Any unbound reporter particle, 107, present in the composition, 313, is then removed, 106, thereby providing a composition, 315, comprising an analyte-reporter particle/capture particle complex, and optionally, unbound capture particle. The [A-RP]/[CP] complex is then detected, 314, by methods described herein.

Referring to FIG. 3, contacting, 104, the sample with a reporter particle, 103, can occur before or after the contacting, 204, with the magnetic capture particle, 203. In other embodiments, the sample can be contacted with the reporter particle and the magnetic capture particle at about the same time. Suitably, the reporter particle that is not bound to the analyte is removed, 106, via washing as described herein and known in the art.

Referring to FIG. 3, the methods can optionally comprise separating, 310, an unbound analyte, 309, from one or more of the compositions. The separating, 310, can comprise applying a magnetic field to the composition, chromatographically separating, contacting a sample with a resin, filtering the sample, centrifuging the sample, and the like, and combinations thereof.

Referring to FIG. 3, prior to contacting with a reporter particle and capture particle, the sample, 101, can be optionally contacted, 150, with a target probe, 151 (TP). In the presence of an analyte, a composition, 153, is provided comprising an analyte-target probe complex [A-TP] and unbound target probe. The unbound target probe, 155, can be optionally removed, 152, from the sample, and the process can be resumed, 154, as described above, except that a reporter particle and/or capture particle can bind to an analyte or the target probe.

Referring to FIG. 3, unbound capture particle, 207, can be optionally separated, 306, from the composition, 315, thereby providing a composition, 317, comprising an analyte bound to a reporter particle and complexed with a capture particle. The presence of the complex is then detected by methods described herein. The separating, 306, can be performed by methods described herein.

In some embodiments, the present invention comprises a process for detecting a nucleic acid analyte, the process comprising contacting a sample comprising one or more nucleic acids with a magnetic capture particle comprising a first oligonucleotide complementary to a first nucleic acid sequence of the analyte, wherein in the presence of a nucleic acid analyte having a nucleotide sequence complementary to the nucleotide sequence of the first oligonucleotide, an analyte-capture particle complex is formed. Unbound capture particles are then optionally removed from the sample. The sample is also contacted with a target probe comprising a second oligonucleotide complementary to a second nucleic acid sequence of the analyte, wherein in the presence of a nucleic acid analyte having a nucleotide sequence complementary to the nucleotide sequence of the second oligonucleotide, an analyte-target probe complex is formed. The sequences of the first and second oligonucleotides are different. The contacting of the sample with the target probe can be performed prior to, after, or simultaneously with, the contacting of the sample with the magnetic capture particle. In some embodiments, the target probe comprises a non-magnetic particle portion (e.g., having at least two different binding groups on a surface thereof, such as an oligonucleotide and a biotin). In some embodiments, a complex comprising a nucleic acid analyte bound to both a target probe and a magnetic capture particle (i.e., TP-A-CP) is formed. Unbound target probe (i.e., target probe that does not bind with an analyte) can be optionally removed from the sample. The sample is then contacted with a reporter particle comprising a plurality of binding moieties capable of binding with the target probe. In the presence of the target probe bound to an analyte, the reporter particle binds a plurality of target probe species and thereby facilitates the aggregation of the magnetic capture particles. In some embodiments, the reporter particle comprises a plurality of avidin binding groups (e.g., streptavidin), which bind, for example, with a biotinylated target probe. The unbound reporter particles are removed from the sample. The degree of complexation (and aggregation) in the sample can then be determined by methods described herein (e.g., by determining a T2 relaxation time of the sample), wherein the degree of complexation (and aggregation) relates directly to the concentration of the target nucleic acid in the sample.

Alternatively, a detector moiety can be added to the sample, wherein the detector moiety comprises one or more binding groups capable of binding to the reporter particle and/or the magnetic capture particle. The degree of aggregation in the sample can then be determined as described herein.

Alternatively, the reporter particles are then disassociated from the complexes. For example, the bond formed by the binding group on the reporter particle with the target probe can be disrupted. Alternatively, a bond linking the binding group of the target probe that is bound to the reporter particle is disrupted, thereby providing unbound reporter particles having a portion of the binding moieties thereon occupied with binding groups from the target probe. The unbound reporter particles that were disassociated from the complexes are then contacted with detector moieties, and in the reporter particles facilitate aggregation of the detector moieties. The degree of aggregation within the sample can be detected by measuring a property of the sample (as described herein).

A property of a sample comprising a nucleic acid analyte that binds to both the target probe and the magnetic capture particle differs from a property of a sample lacking this analyte because the concentration of reporter particles able to participate in the aggregation with the detector moieties relates directly to the concentration of the analyte in the sample. Alternatively, a detector moiety can be added directly to a sample while the reporter particle is bound to the complex. In either case, the present invention provides a method for detecting the presence of a target nucleic acid in a sample without requiring amplification of the desired nucleic acid sequence. Not being bound by any particular theory, the aggregation of the magnetic capture particles amplifies the presence of the target nucleic acid, thereby rendering it detectable using a laboratory bench-top apparatus without the need for enzymatic amplification of the target nucleic acid.

In some embodiments, the present invention is directed to a method comprising contacting a sample comprising one or more analytes selected from: a protein, a saccharide, an infectious agent, a cell, and a combination thereof, with a capture particle comprising an antibody binding group capable of specifically binding to a first site on the analyte. Unbound capture particles are optionally removed from the sample. The sample is then contacted with a target probe comprising a second binding group capable of binding specifically with a second site on the analyte. For example, the second binding group can comprise a small molecule capable of binding with an active site of a protein, an infectious agent, and/or a cell surface. Other suitable second binding groups include, but are not limited to, metals (e.g., metal ions), antibodies, and the like. After contacting with the target probe, the sample can be optionally treated (e.g., washed) to remove unbound target probe from the sample. The sample is then contacted with a reporter particle capable of binding to a third binding group on the target probe. Unbound reporter particle is removed from the sample, and the presence of the reporter particle in the sample is detected by methods described herein. Specifically, the degree of aggregation in the sample can be determined directly, or a detector moiety can be added to the sample and the degree of aggregation of the detector moiety with the complexes in the sample can be used to determine a property of the sample.

Alternatively, the reporter particles can be disassociated from the complexes, isolated, and then contacted with detector moieties, wherein the degree of aggregation of the reporter particles with the detector moieties is used to determine a property of the sample. In all cases, the degree of aggregation in the sample is a direct, sensitive, quantitative measurement of the analyte concentration in the initial sample.

### Complexes

The present invention is also directed to a complex comprising an analyte, a magnetic capture particle comprising a first binding group bound to a first site on the analyte by a first specific binding interaction, a target probe comprising a second binding group bound to a second site on the analyte by a second specific binding interaction, wherein the first and second binding groups are different, and a reporter particle comprising a plurality of binding groups bound to a third binding group on the target probe, wherein the second and third binding groups are different.

In some embodiments, a magnetic capture particle present in a complex comprises a superparamagnetic particle having a cross-sectional dimension of 50 nm to 20 μm, 100 nm to 15 μm, or about 1 μm in size. In some embodiments the reporter particle has a plurality of biotin molecules on its surface, and the reporter particle is bound to the target probe via a biotin-avidin interaction.

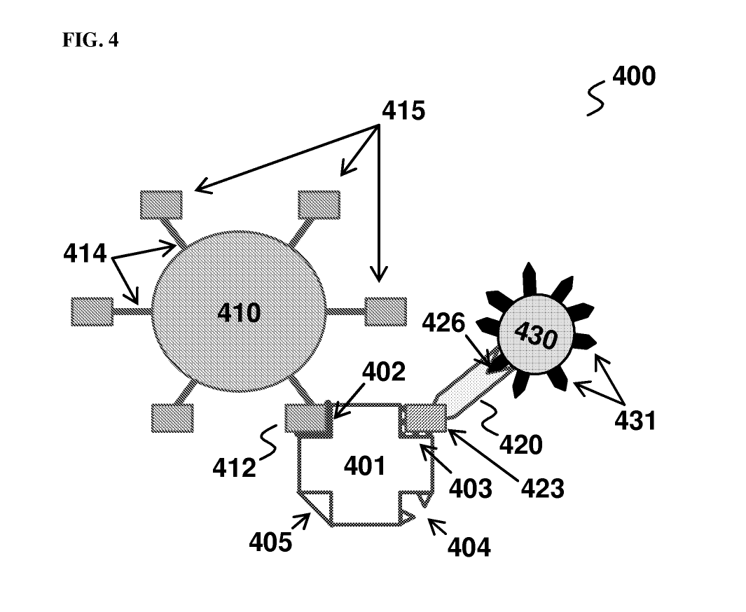

FIG. 4 provides a schematic cross-sectional representation of a complex of the present invention. Referring to FIG. 4, a complex, 400, is provided, the complex comprising an analyte, 401, and a magnetic capture particle, 410, comprising a first binding group, 412, that is bound to a first site, 402, of the analyte by a specific binding interaction. In some embodiments, the magnetic capture particle comprises a linker, 414, connecting the capture particle, 410, with the first binding group, 412. In some embodiments, the capture particle, 410, comprises a plurality of binding groups, 415, on its surface. However, it is not necessary that all the binding groups be specifically bound to sites on an analyte. The plurality of binding groups, 415, can be the same or different than the first binding group, 412. The complex also comprises a target probe, 420, comprising a second binding group, 423, bound to a second site, 403, on the analyte, 401. The first binding group, 412, and second binding group, 423, arc different and specifically bind to different regions or sites on the analyte. In embodiments in which the analyte is, for example, an ion, the first and second binding groups can be, for example, monodentate or polydentate ligands that bind to the ion simultaneously. In embodiments in which the analyte is a protein, a nucleic acid, a saccharide, a lipid, a small molecule, a gas, an infectious agent, and/or a cell, the binding can be in distinctly different sites or regions of the analyte. The complex also comprises a reporter particle, 430, comprising a plurality of binding groups, 431. The reporter particle, 430, is bound to the target probe, 420, via a specific bonding interaction between one or more of the binding groups, 431, and a binding/active site, 426, on the target probe.

The present invention is also directed to a complex comprising a nucleic acid, a magnetic capture particle comprising a first oligonucleotide bound to a first sequence of the nucleic acid by a nucleotide base-pairing interaction, a target probe comprising a second oligonucleotide bound to a second sequence of the nucleic acid by nucleotide base-pairing interaction, wherein the first and second sequences of the nucleic acid are different, and a reporter particle comprising a plurality of binding groups bound to the target probe. The base pairing interactions between the nucleic acid and the first oligonucleotide and the target probe and the second oligonucleotide can comprise 3 to about 40 base-pairings per binding interaction. The complex comprises a reporter particle bound to the target probe, wherein the reporter particle comprises a plurality of binding groups on its surface. In some embodiments, the reporter particle and target probe bind to each other by an avidin-biotin interaction, a nucleotide base-pairing interaction, and the like.

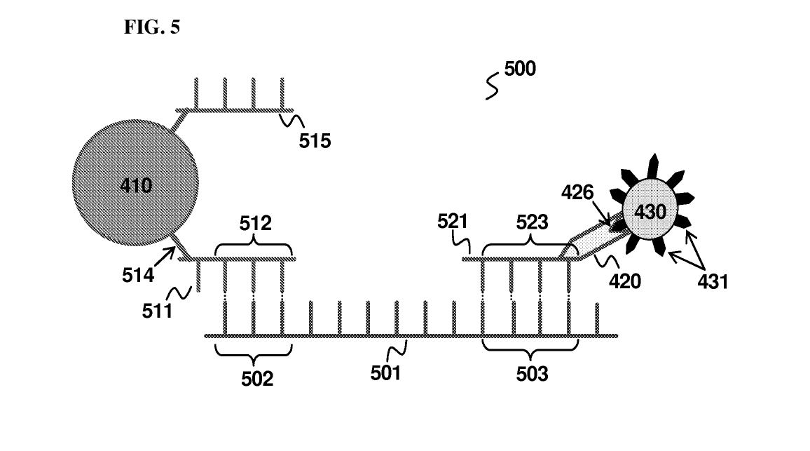

FIG. 5 provides a schematic cross-section representation of a complex of the present invention. Referring to FIG. 5, a complex, 500, is provided, the complex comprising a nucleic acid, 501, and a magnetic capture particle, 410, comprising a first oligonucleotide, 511, that is bound to a first sequence, 502, of the nucleic acid by a nucleotide base-pairing interaction. For example, the sequence, 512, of the first oligonucleotide, 511, is complementary to the first sequence, 502, of the nucleic acid. In some embodiments, the capture particle comprises a linker, 514, connecting the capture particle, 410, with the first oligonucleotide, 511. As depicted in FIG. 5, it is not necessary for the entire sequence of the first oligonucleotide to participate in the nucleotide base-pairing interaction. In some embodiments, the capture particle, 510, includes an optional second (or more) oligonucleotide(s), 515, attached thereto, wherein the second oligonucleotide can have a sequence the same or different than the sequence of the first oligonucleotide, 511. The complex also comprises a target probe, 420, comprising a second oligonucleotide, 521, bound to a second sequence, 503, of the nucleic acid, 501. For example, the sequence, 523, of the second oligonucleotide, 511, is complementary to the second sequence, 503, of the nucleic acid. The first sequence, 502, and the second sequence, 503, of the nucleic acid, 501, are different. The complex also comprises a reporter particle, 430, comprising a plurality of binding groups, 431. The reporter particle, 430, is bound to the target probe, 420, via a specific bonding interaction between one or more of the binding groups, 431, and a binding/active site, 426, on the target probe.

Although not shown in FIGS. 4-5, a complex of the present invention can comprise multiple analytes bound to an individual capture particle, multiple target probes (bound to analytes) that are bound to an individual reporter particle, and combinations thereof. Thus, in some embodiments the complexes of the present invention are agglomerates. It is not necessary that every analyte present in an agglomerate of the present invention be bound to both a capture particle and a target probe, so long as at least a portion of the analytes present in the sample are bound to both a capture particle and a target probe.

Not being bound by any particular theory, the complexes of the present invention can form highly cross-linked agglomerates that are readily separable from a sample using methods described herein such as, but not limited to, magnetic separation methods. Thus, the complexes of the present invention provide a significant advancement over previously described analyte complexes because there is no need to amplify the analyte prior to forming a complex prior to detection. Instead, the complexes can be directly detected (by methods described herein) with a high degree of quantitative sensitivity.

### Reagent Cartridges

The present invention is also directed to a reagent cartridge comprising a plurality of wells, each well suitable for holding a sealable container at a predetermined position, wherein the cartridge comprises a first sealable container at a first position that includes a reporter particle comprising a plurality of binding groups capable of binding to an analyte; and a second sealable container at a second position that includes detector moiety, wherein the detector moiety is magnetic, fluorescent, radioactive, or a combination thereof.

A reagent cartridge can have any dimension suitable for interfacing with an analytical device suitable for carrying out the methods of the present invention. The reporter particles and detector moieties are those described herein. The reagents (e.g., the reporter particles and detector moieties) are present in an amount sufficient for carrying out one or more analyses of a sample, or a plurality of samples. The containers are sealable, and in some embodiments are resealable. For example, a container can include a resealable surface such as a lid, a cap, and the like, or a pierce-able surface such as a membrane, a foil surface, and the like. In some embodiments, the sealable container is substantially impermeable to oxygen, or has an oxygen permeability of 1×10⁻¹¹ cc·cm/cm²·sec·cm Hg or less, or 1×10⁻¹² cc·cm/cm²·sec·cm Hg or less.

Having generally described the invention, a further understanding can be obtained by reference to the examples provided herein. These examples are given for purposes of illustration only and are not intended to be limiting.

### EXAMPLES

The following examples are illustrative, but not limiting, of the method and compositions of the present invention. Other suitable modifications and adaptations of the variety of conditions and parameters normally encountered in nanocrystal synthesis, and which would become apparent to those skilled in the art, and are within the spirit and scope of the invention.

### Example 1

### Non-Enzymatic Detection of Nucleic Acids

### Generation of Streptavidin Functionalized Reporter Particles

A. Two different covalent chemistries were employed to conjugate oligonucleotides to streptavidin (SA). First, a bifunctional crosslinker (sulfo-SMCC) was conjugated onto solvent accessible amines in streptavidin following the protocol in Current Protocols in Nucleic Acid Chemistry 12.7.1-12.7.15 (2005). This conjugation yielded a maleimide-activated streptavidin that can then be covalently conjugated to thiolated oligonucleotides. Thiolated protected oligonucleotides identical to a lambda 708 sequence (complementary to the sense strand of lambda phage genome (from nucleotide 708-743)) were obtained from Integrated DNA Technologies and deprotected with DTT prior to conjugation.

SEQ ID NO 1:
TCA GCC TGT TAA CCT GAC TGT TCG ATA TAT TCA

Several distinct bands on a native acrylamide gel were visible when the gel was stained with nucleic acid specific SYBR gold stain. A single band migrating approximately 4 cm into the gel which stained with both a protein-specific stain (Coomassie Blue) and a nucleic acid specific stain (SYBR green) was purified. Absorption spectroscopy confirmed that the complex contained a 1:1 ratio of oligonucleotide to SA tetramer. The biotin binding capacity of the oligo-SA was tested by binding to biotinylated particles, then hybridizing a Cy5 complement to the bound oligo. The Cy 5 oligo was heat dissociated and quantified using fluorescence detection. Measured binding capacity was low: ˜40 pmoles/mg particles.

In a second covalent conjugation approach, maleimide activated streptavidin (Pierce) was obtained for conjugation directly to thiolated oligonucleotides. Though the manufacturer indicated each streptavidin contained a single maleimide, when examined on native acrylamide gel, the presence of multiple bands indicated either streptavidin tetramer dissociation and/or multiple sites of maleimide conjugation.

Protein/oligonucleotide conjugates were also prepared by binding biotinylated oligonucleotides complementary to the sense strand of lambda phage genome (from nucleotide 708-743) to streptavidin. The oligo-conjugates were then gel purified.

SEQ ID NO 2:
TCA GCC TGT TAA CCT GAC TGT TCG ATA TAT TCA

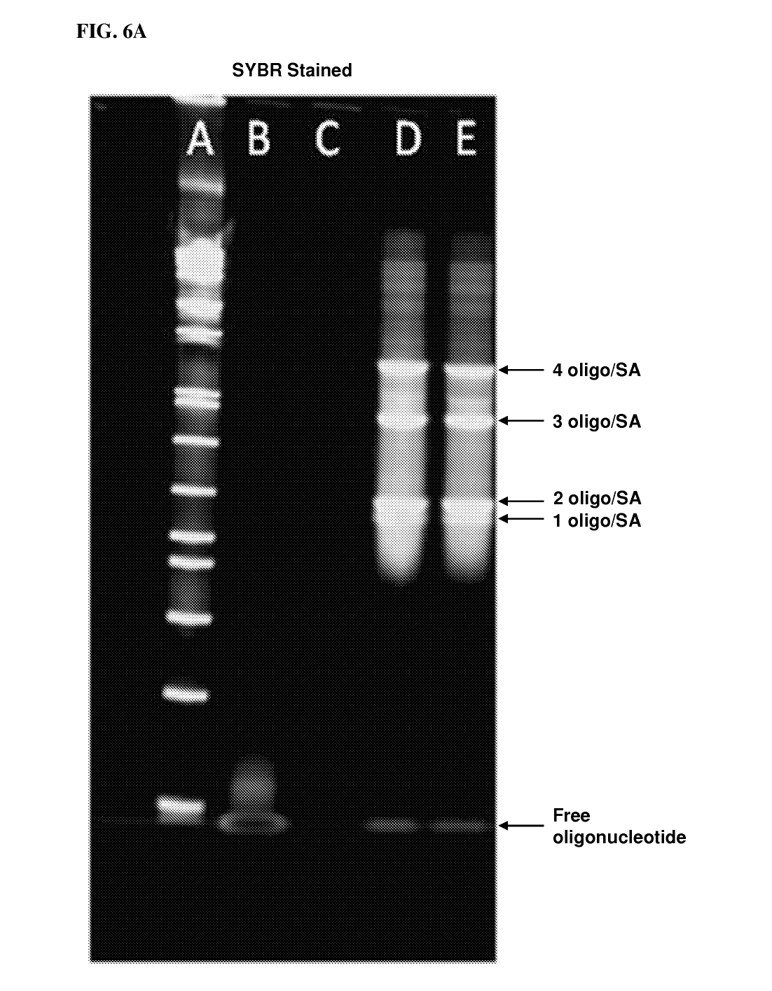

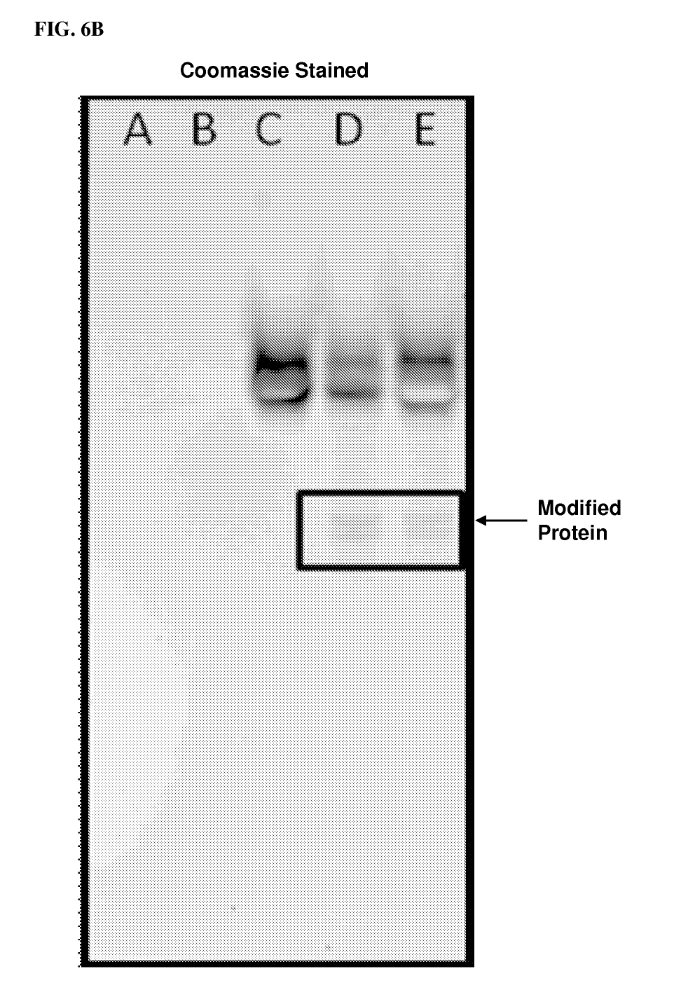

Briefly, ˜10 nmoles of a 5′ biotinylated oligonucleotide identical to lambda 708 sequence was bound to 10 nmoles of purified streptavidin (Roche, Indianapolis, Ind.) in a 30 μL reaction in TE (pH 8). Prepared conjugates were purified by acrylamide gel electrophoresis, then excised bands eluted in Tris-glycine buffer. Biotin binding capacity of resulting oligonucleotide conjugates were measured by binding oligonucleotide conjugate to biotinylated reporter particles, then hybridizing a Cy5 labeled complement to the immobilized oligonucleotides. A biotin binding capacity of ˜600 pmoles/mg particles was detected with non-covalently bound, purified complexes. This represented the highest biotin binding capacity of any of the prepared conjugation methods. FIGS. 6A-6B depicts gel images resulting when non-covalent conjugates were electrophoresed on a native gel and stained with SYBR gold (FIG. 6A; specific staining for nucleic acid) and Coomassie blue (FIG. 6B; specific staining for streptavidin protein).

Referring to FIGS. 6A-6B, lane A is a 1 kb MW ladder (INVITROGEN®). Lane B is an aliquot of free 43-mer oligonucleotide. Lane C is free streptavidin. Lane D is a conjugation reaction of 10 nmoles of biotinylated oligonucleotide/10 nmoles of streptavidin tetramer (1:1 ratio of oligo/SA). Lane E is a conjugation reaction of 10 nmoles of biotinylated oligonucleotide/40 nmoles of streptavidin (1:4 ratio of oligo/SA). The lower doublet consisting of single and dual oligo bound streptavidin was excised from the gel and electroeluted.

Since the highest biotin binding capacity was achieved with prepared non-covalent conjugates, a large scale binding reaction was prepared (˜10 mg streptavidin) for FPLC purification on a MonoQ HR5/5 anion exchange column (GE Lifesciences, Piscataway, N.J.). A buffer gradient used for purification is given in Table 1. FPLC purification was conducted at Excellgen, Inc. (Gaithersburg, Md.). Received fractions were subjected to repeat absorption spectroscopy measurement and the single oligo bearing fractions were pooled and concentrated for further use.

TABLE 1
Buffer gradient used for FPLC purification of oligo-streptavidin
conjugates

| Volume (mL) | [NaCl] (M) | Flow Rate (mL/min) |
|---|---|---|
| 0-4 min | 0.3 | 0.2 |
| 4-8 min | 0.3 + 0.015/min | 0.2 |
| 8-40 min | 0.45 + 0.01/min | 0.2 |

### Generation of Covalent Oligonucleotide-Capture Particle Conjugates

DNA oligonucleotides were procured from Integrated DNA Technologies (Coralville, Iowa) for conjugation to Magnetic Capture Particles. Aminated oligonucleotides were standard desalt purified, and oligonucleotides longer than 50 nucleotides in length were PAGE purified. Oligonucleotide purity was measured at IDT via mass spec and capillary electrophoresis.

35-mer oligonucleotide functionalized at the 5′ end with an amino group:

(SEQ ID NO: 3)
5′-TTT GAT GAT ATC CCG TTT CAG GAA ATC AAC
ATG TC-3′.

The oligo sequence was complementary to the sense strand of lambda phage genome (from nucleotide 628 to 663).

Prior to coupling, the stock particle suspension was prepared by vortexing and visual inspection to eliminate any pellet or particle clumping, then the particles were washed three times with deionized water and then resuspended in 30 μL of deionized water. Successful conditions for coupling oligo to SERADYN® 1 μM carboxy-functionalized particles include, for example, the following: a) washed particles were resuspended in 30 μL water, and added to a solution comprising sterile deionized water (46 μL), 500 mM MES (10 μL), and amine-modified oligo (1 nmol/μL, 4 μL); b) following this 5 minute pre-incubation, freshly prepared N-Ethyl-N′-(3-dimethylaminopropyl) carbodiimide hydrochloride (EDAC) was added to a final concentration that was 1% (w/v) of the final reaction volume; c) a 10% w/v stock solution (10 μL) was added to our 90 μL of bead slurry (in a final 50 mM MES solution); d) conjugation reactions were incubated overnight at 37° C., with mixing. After conjugation the particles were subjected to two 5 minute de-ionized water washes at room temperature, two 5 minute 0.1 M imidazole (pH 6) washes at 370° C., three 5 minute 0.1 M sodium bicarbonate washes at 37° C., and two 30 minute sodium bicarbonate washes at 65° C. The Magnetic Capture Particles were then stored as 1% suspensions in TE (pH 8) 0.1% TWEEN® (Uniqema Americas LLC).

### Streptavidin Functionalized Detector Moieties (Particles)

DYNABEADS® MYONE™ Streptavidin-C1 coated 1 μm superparamagnetic particles were purchased from INVITROGEN® (INVITROGEN DYNAL® AS, Oslo, Norway).

### Generation of Biotinylated Reporter Particles, and Detection of Particles

High lot-to-lot variability was present among purchased biotinylated particle production lots, with streptavidin binding capacities varying by as much as 3-fold, and assay detection sensitivity changes of 2-logs (from 10³ to 10⁵) with lower binding capacity particles. Thus, we produced our own biotinylated reporter particles. Aminated biotin (N-(2-aminoethyl) biotinamide and N-(5-aminopentyl)biotinamide (INVITROGEN®) were conjugated to a variety of carboxylated polystyrene particles (see Table 2 below). Sulfo-succinimydal ester biotin (INVITROGEN®) was also conjugated to aminated carboxylated 1 μm polystyrene particles (INVITROGEN®).

T2 detection sensitivity for the various biotinylated reporter particles was measured by combining the biotinylated reporter particles with MyOne streptavidin-coated paramagnetic detector particles (INVITROGEN®) in an agglomeration reaction. The results are listed in Table 2.

For example, biotin/streptavidin binding reactions were conducted in PBS, 0.1% BSA, and 0.1% TWEEN® (Uniqema Americas LLC) at a volume of 30 μL. Streptavidin-coated paramagnetic detector particles (MyOne™ 1 μm streptavidin-coated particles, INVITROGEN®) were present at 3×10⁶ particles/reaction. Binding reactions were incubated with agitation (about 600-1000 rpm) at 40° C. within a Vortemp heated shaker for 20 minutes. Reactions were then diluted to 150 μL in PBS with 0.1% BSA and 0.1% TWEEN® (Uniqema Americas LLC), and incubated under magnetic field for 10 minutes. Samples were then briefly vortexed and subjected to T2 measurements using a BRUKER® minispec. For comparison purposes the detection sensitivity measured for two new lots of purchased INVITROGEN® biotinylated particles are shown in the first two entries of the table. Highest detection sensitivity was observed when a Bangs 900 nm high acid carboxylated particle was conjugated to ethylenediamine biotin. Approximately 5,000 particles in a 150 μL reaction volume were detectable.

TABLE 2
Detection Sensitivity of Various Reporter Particles

| Reporter Particle | Detector Particle | Best LoD |
|---|---|---|
| INVITROGEN ® Fluosphere 1 μm, bt | MYONE ™ C1, SA | 1.0E+05 |
| INVITROGEN ® Fluosphere 1 μm, | MYONE ™ C1, SA | 1.0E+05 |
| fluorescent, bt | | |
| Bangs 7740 1 μm, 708-bt | MYONE ™ C1, SA | 1.0E+06 |
| Bangs 2 μm, 708-bt | MYONE ™ C1, SA | 1.0E+06 |
| POLYSCIENCES, INC. ® 2 μm, SA | MOBX1 (bt) | 1.0E+06 |
| INVITROGEN ® Fluosphere amino, | MYONE ™ C1, SA | 1.0E+05 |
| B6352 bt | | |
| INVITROGEN ® Fluosphere amino, | MYONE ™ C1, SA | 1.0E+05 |
| B6353 bt | | |
| Bangs 7740 1 μm, A1593 bt | MYONE ™ C1, SA | 1.0E+04 |
| Bangs 7740 1 μm, A1594 bt | MYONE ™ C1, SA | 1.0E+04 |
| INVITROGEN ® Fluosphere COOH, | MYONE ™ C1, SA | 5.0E+03 |
| A1593 bt | | |
| INVITROGEN ® Fluosphere COOH, | MYONE ™ C1, SA | 5.0E+03 |
| A1594 bt | | |
| SERADYN ® 500 nm, A1593 bt | MYONE ™ C1, SA | 1.0E+06 |
| Bangs 6499 900 nm, A1593 bt | MYONE ™ C1, SA | 1.0E+03 |

### Sample Preparation: DNA Shearing

Provided methods have a targeted turn-around time of 60 minutes, which requires a relatively short hybridization time (e.g., about 30 minutes). Intact mega-plasmid or genomic DNA has a radius of gyration that is on the order of microns, which correlates to extremely slow hybridization times due to the slow relative diffusion rate of the large DNA within the constraining matrix generated by the micron-sized magnetic capture particles and reporter particles. Thus, a requirement of the current nucleic acid assay can be that any sample DNA be sheared prior to loading. In some embodiments, DNA samples require shearing to a size of <2000 bp to allow for rapid hybridization.

Many available DNA fragmentation methods are known and available, including enzymatic digestion, mechanical shearing induced by sonication atomization, nebulization, and point-sink shearing. See, e.g., Deininger, P. L., Anal. Biochem. 129:216-223 (1983); Cavalieri, L. F., et al., J. Am. Chem. Soc. 81:5136-5139 (1959); Bodenteich, A., S. et al., “Shotgun cloning as the strategy of choice to generate templates for high throughput dideoxynucleotide sequencing in Automated DNA sequencing and analysis techniques” (ed. M. D. Adams, C. Fields, and C. Venter), pp. 42-50 (Academic Press, London, UK, 1994); and Oefner, P. J., et al., Nucleic Acids Res. 24:3879-3886 (1996). Bench-top and handheld devices are available for preparation of fragmented DNA, including, for example: the GeneMachine from DigiLab Genomic Solutions, a point-sink shearing device capable of processing samples in volumes ranging from 40 uL to 500 μL which has a small footprint (5″ W×10″ D×12″ H), and can fragment down to ˜2 kb; and the S2 instrument from COVARIS® (Woburn, Mass.), which uses a tunable adaptive acoustic focusing device to disrupt both cells and double stranded DNA, and offers precise control of generated fragment sizes. Sheared DNA samples can also be prepared using a sonicator probe.

### Assay Method

Streptavidin functionalized reporter particles prepared as above, oligonucleotide-conjugated magnetic capture particles prepared as above, biotinylated reporter particles prepared as above, and streptavidin functionalized detector moieties described above were used to conduct nucleic acid detection assays using serially diluted lambda oligonucleotide. Prepared target probes, i.e., particles functionalized with both oligonucleotides and streptavidin (oligo-RP-SA) were diluted in PBS to a concentration of 3.3×10¹¹ copies/4; the prepared oligonucleotide-functionalized magnetic capture particles (oligo-MCPs) were diluted to 1×10⁶ particles/4 in TE, 0.1% TWEEN® (Uniqema Americas LLC); and the prepared biotinylated reporter particles (biotin-RPs) were diluted to a final concentration of 1.5×10⁷ particles/4 in TE, 0.1% TWEEN®-20.

Briefly, target DNA (lambda 628-T18-708 oligonucleotide, Integrated DNA Technologies, Inc.) was subjected to 10-fold serial dilutions in TE (pH 8) at copy numbers spanning 1×10¹¹ copies/4 to 1×10² copies/4 in the final reactions, and then contacted with the target probes (i.e., oligo-RP-SA, 1×10¹² copies) and oligo-MCPs (3×10⁶ copies) to conduct hybridization reactions in 2×SSC, 0.1% TWEEN®-20, 2.5% formamide, and 10 μg sheared salmon sperm DNA. The hybridization reaction samples were denatured at 70° C. for 3 minutes with agitation, followed by hybridization at 40° C. for 90 minutes with agitation. Following hybridization, the samples were subjected to magnetic separation, whereby the samples were washed twice in 1×SSC to remove unbound target probes and unbound capture particles, and then resuspended in 1×SSC with 0.1% TWEEN®-20 (18 μL). 2 μL of the diluted biotin-RPs (3×10⁷ copies) was then added to the sample comprising the complexes (i.e., [MCP-oligo]-[target DNA]-[oligo-RP-SA] complexes) and allowed to bind with the streptavidin binding group on the target probes present in the complexes. The binding was allowed to proceed by incubating for one hour at 30° C. with agitation.

Following binding, the samples were subjected to magnetic separation, whereby the samples were washed twice in 1×SSC. The SA-functionalized reporter particles that were previously bound to the complexes were then disassociated from the complexes by resuspension of the samples in 0.2 N NaOH (20 μL) with incubation at room temperature for ten minutes. The samples were again subjected to magnetic separation, and the unbound reporter particles were collected for detection.

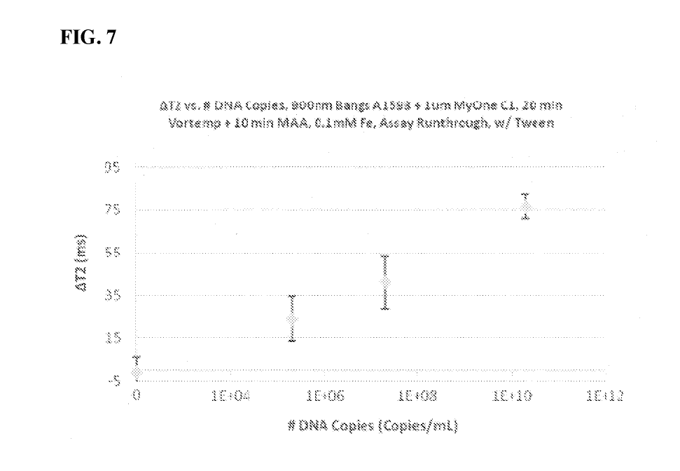

For the detection phase, 10 μL, of the unbound reporter particles that were disassociated from the complexes were combined with 10 μL TE, and 10 μL of prepared detector moieties (streptavidin-functionalized particles (DYNABEADS® MYONE™ Streptavidin-C 1 coated 1 μm superparamagnetic particles, INVITROGEN DYNAL® AS, Oslo, Norway), at a concentration of 3×10⁵ particles/4 in TE, 0.1% TWEEN® (Uniqema Americas LLC) for a final concentration of 3×10⁶ particles/30 μL reaction. The reaction was incubated for 20 minutes at 40° C. with agitation. The samples were then diluted to 150 μl with PBS/0.1% BSA/0.1% TWEEN®-20, transferred to a borosilicate glass NMR tube, placed in a homogeneous magnetic field (e.g., in a BRUKER® mini-spec magnet) for 10 minutes at 40° C. Samples were then briefly vortexed and subjected to T2 measurements using the BRUKER® minispec. The program parameters utilized for obtaining T2 measurements in the BRUKER® mini-spec are shown in Table 3. Exemplary results are depicted in FIG. 7, depicted as delta T2 (i.e., background T2 measurements are subtracted from T2 values) values, with each data point representing a mean of n=2±SD. Results indicated detection of nucleic acid target above 1×10⁵ to 1×10⁶ copies per mL.

TABLE 3
Program Parameters Used for Relaxation Measurements

| | |
|---|---|
| # Scans: | 1 |
| Recycle delay: | 1.00 |
| Inter-echo delay: | 0.5 |
| Tau: | 0.25 |
| # Echoes collected: | 3000 |
| # Dummy echoes collected per collected echo: | 2 |
| Total echo train time: | 4500 |
| Receiver gain: | 76 |

### CONCLUSION

Exemplary embodiments of the present invention have been presented. The invention is not limited to these examples. These examples are presented herein for purposes of illustration, and not limitation. Alternatives (including equivalents, extensions, variations, deviations, etc., of those described herein) will be apparent to persons skilled in the relevant art(s) based on the teachings contained herein. Such alternatives fall within the scope and spirit of the invention.

It is to be appreciated that the Detailed Description section, and not the Summary and Abstract sections, is intended to be used to interpret the claims. The Summary and Abstract sections can set forth one or more, but not all exemplary embodiments of the present invention as contemplated by the inventor(s), and thus, are not intended to limit the present invention and the appended claims in any way.

All publications, patents and patent applications mentioned in this specification are herein incorporated by reference to the same extent as if each individual publication, patent or patent application was specifically and individually indicated to be incorporated by reference.

1. A method of detecting one or more analytes in a sample, the method comprising:
(i) contacting the sample with a reporter particle capable of binding to the one or more analytes, wherein in the presence of an analyte, the reporter particle binds to the analyte;
(ii) removing unbound reporter particle from the sample;
(iii) following step (ii), contacting the sample with a detector moiety, wherein in the presence of the remaining reporter particle, the detector moiety forms an agglomerate; and
(iv) detecting the analyte in the sample by measuring a value of a property of the agglomerate, wherein the value of a sample comprising the one or more analytes differs from the value of a reference sample lacking the one or more analytes.

2. The method of claim 1, wherein the detector moiety is magnetic, light-absorptive, fluorescent, chiral, radioactive, or a combination thereof.

3. The method of claim 1, wherein the detecting comprises measuring a property of the sample selected from: a nuclear magnetic resonance property, a relaxation time, an ultraviolet absorption, a visible absorption, a fluorescence intensity, a fluorescence decay time, a circular dichroism, a radioactive half-life, a radioactive emission signal, a turbidity, a density, and combinations thereof.

4. The method of claim 1, wherein the detecting comprises determining a relaxation time of the sample by magnetic resonance spectroscopy.

5. The method of claim 4, wherein the detecting comprises determining a T2 relaxation time.

6. The method of claim 1, wherein the reporter particle comprises a non-magnetic reporter particle that includes a plurality of binding groups.

7. The method of claim 1, wherein the detector moiety comprises a binding group capable of binding to the analyte, and wherein in the presence of the analyte, the detector moiety binds to the analyte.

8. The method of claim 1, wherein the detector moiety comprises a binding group capable of binding to the remaining reporter particle, and wherein in the presence of the remaining reporter particle, the detector moiety binds to the remaining reporter particle.

9. The method of claim 8, comprising disassociating the bound reporter particle from the analyte following step (ii) and prior to the step (iv).

10. The method of claim 9, wherein the disassociating comprises a process selected from: temperature denaturing, generating a pH gradient, reducing disulfide bonds, oxidizing disulfide bonds, mechanically disrupting, and combinations thereof.

11. The method of claim 1, wherein the detector moiety comprises a magnetic particle, and wherein in the presence of the reporter particle an agglomerate of the magnetic particles is formed.

12. The method of claim 1, further comprising contacting the sample with a target probe, the target probe comprising a first binding group capable of binding to a first target site on the analyte and a second binding group capable of binding to the reporter particle or the detector moiety, wherein in the presence of one or more analytes, the target probe binds to the first target site on the analyte via the first binding group and binds to the reporter particle or the detector moiety via the second binding group.

13. The method of claim 12, comprising disassociating the target probe from the analyte by disrupting the specific binding between the first binding group and first binding site.

14. The method of claim 1, wherein the removing comprises washing the sample to remove reporter particles that are not bound to the analyte.

15. The method of claim 1, wherein the reporter particle or the detector moiety is paramagnetic and the method comprises subjecting the sample to magnetic assisted agglomeration prior to the detecting.

16. The method of claim 1, wherein the analyte is selected from: a protein, a nucleic acid, a saccharide, a lipid, a small molecule, an ion, a gas, an infectious agent, a cell, and combinations thereof.

17. A method of detecting one or more analytes in a sample, the method comprising:
(a) contacting the sample with a capture particle comprising a first binding group capable of specifically binding to a first binding site on the one or more analytes, wherein in the presence of an analyte, the capture particle binds to the first binding site;
(b) contacting the sample with a reporter particle comprising a plurality of binding groups capable of binding to the analyte-capture particle complex, wherein in the presence of the analyte, the reporter particle binds to the analyte-capture particle complex;
(c) following step (b), removing unbound reporter particle from the sample; and
(d) detecting the presence of the reporter particle.

18. The method of claim 17, further comprising, following step (c), disassociating bound reporter particle from the analyte prior to the detecting.

19. The method of claim 18, wherein the disassociating comprises releasing the reporter particle from the analyte-capture particle complex by disrupting a specific binding interaction between the reporter particle and the analyte.

20. The method of claim 18, wherein the disassociating comprises releasing the reporter particle from the analyte-capture particle complex by disrupting a specific binding interaction between the reporter particle and the capture particle.

21. The method of claim 18, wherein the disassociating comprises a process selected from: temperature denaturing, generating a pH gradient, reducing disulfide bonds, oxidizing disulfide bonds, mechanically disrupting, and combinations thereof.

22. The method of claim 18, further comprising, prior to the detecting, contacting the disassociated reporter particle with a detector moiety to form an aggregate of the reporter particle and the detector moiety, wherein the detecting comprises measuring a value of a property of the aggregate, wherein the value of a sample comprising the one or more analytes differs from the value of a reference sample lacking the one or more analytes.

23. The method of claim 22, wherein the detector moiety comprises a plurality of avidin-functionalized binding groups capable of binding to the disassociated reporter particle via a biotin-avidin interaction.

24. The method of claim 17, wherein the analyte comprises a nucleic acid, and wherein the first binding group comprises a first oligonucleotide capable of specifically binding to a first nucleic acid sequence on the analyte via a specific nucleotide base-pairing interaction with the first nucleic acid sequence.

25. The method of claim 17, wherein the analyte is selected from: a protein, a saccharide, an infectious agent, a cell, or a combination thereof, and wherein the first binding group comprises an antibody capable of specifically binding to the first binding site.

26. The method of claim 17, comprising contacting the sample with a target probe, the target probe comprising a second binding group capable of specifically binding to at least the analyte or the capture particle, wherein the first and second binding groups are different, and wherein in the presence of the analyte, the target probe binds to at least the analyte or the capture particle by a specific binding interaction.

27. The method of claim 26, wherein the second binding group comprises a second oligonucleotide capable of specifically binding to a second binding site on a nucleic acid via a complementary nucleic acid base pairing interaction, and wherein the first and second oligonucleotides are different.

28. The method of claim 26, wherein the second binding group comprises an antibody capable of specifically binding to a second binding site on an analyte selected from: a protein, a saccharide, an infectious agent, a cell, or a combination thereof.

29. The method of claim 17, wherein the reporter particle comprises a plurality of biotin binding groups capable of binding to the target probe via a biotin-avidin interaction.

30. The method of claim 17, wherein the capture particle is magnetic.

31. The method of claim 30, further comprising separating the analyte bound to the magnetic capture particles from the sample using a magnetic field.

32. The method of claim 17, wherein the detecting comprises determining a magnetic resonance relaxation time of the sample.

33. The method of claim 17, further comprising contacting the sample with a target probe, the target probe comprising a second binding group capable of specifically binding to the one or more analytes, wherein the first and second binding groups are different, and wherein in the presence of an analyte, the target probe binds to the analyte by a specific binding interaction;
wherein the analyte comprises a nucleic acid,
wherein the magnetic capture particle comprises a first oligonucleotide complementary to a first nucleic acid sequence of the analyte,
wherein the target probe comprises a second oligonucleotide complementary to a second nucleic acid sequence of the analyte,
wherein the first and second nucleic acid sequences are different, and
wherein the reporter particle comprises a plurality of binding groups capable of binding to the target probe, wherein in the presence of the analyte, the reporter particle binds to the target probe.

34. The method of claim 17, further comprising:
(x) contacting the sample with a target probe, the target probe comprising a second binding group capable of specifically binding to the one or more analytes, wherein the first and second binding groups are different, wherein in the presence of an analyte, the target probe binds to the analyte by a specific binding interaction, and the reporter particle binds to the target probe;
(y) prior to step (b), separating unbound target probe from target probe bound to the analyte-capture particle complex; and
(z) disassociating bound reporter particle from the analyte-capture particle complex prior to the detecting,
wherein the analyte comprises a nucleic acid,
wherein the capture particle is magnetic and comprises an oligonucleotide complementary to a first nucleic acid sequence of the analyte,
wherein the target probe comprises an oligonucleotide complementary to a second nucleic acid sequence of the analyte, and
wherein the first and second nucleic acid sequences are different.

35. The method of claim 34, wherein the reporter particle comprises a plurality of biotin binding groups, capable of binding to a target probe via a biotin-avidin interaction in the presence of an analyte.

36. The method of claim 1, wherein the method has a limit of detection of at least 1×10³ analytes per milliliter of sample.

37. A complex comprising:
(i) an analyte;
(ii) a magnetic capture particle comprising a first binding group bound to a first site on the analyte by a first specific binding interaction;
(iii) a target probe comprising a second binding group bound to a second site on the analyte by a second specific binding interaction, wherein the first and second binding groups are different; and
(iv) a reporter particle comprising a plurality of binding groups bound to a third binding group on the target probe, wherein the second and third binding groups are different.

38. The complex of claim 37, wherein the magnetic capture particle comprises a superparamagnetic particle having a cross-sectional dimension of 50 nm to 20 μm.

39. The complex of claim 37, wherein the reporter particle comprises a plurality of biotin binding groups and binds to the target probe via a biotin-avidin interaction.

40. The complex of claim 37, wherein:
the analyte is a nucleic acid;
the magnetic capture particle comprises a first binding group that is a first oligonucleotide bound to a first sequence of the nucleic acid analyte by a nucleotide base-pairing interaction; and
the target probe comprises a second binding group that is a second oligonucleotide bound to a second sequence of the nucleic acid by nucleotide base-pairing interaction, wherein the first and second sequences of the nucleic acid are different.

41. A reagent cartridge comprising a plurality of wells, each well suitable for holding a sealable container at a predetermined position, wherein the cartridge comprises a first sealable container at a first position that includes a reporter particle comprising a plurality of binding groups capable of binding to an analyte; and a second sealable container at a second position that includes a detector moiety, wherein the detector moiety is magnetic, fluorescent, radioactive, or a combination thereof.
